(19) United States
(12) Patent Application Publication
(10) Pub. No.: US 2009/0041747 A1
(43) Pub. Date: Feb. 12, 2009

(54) Compounds for Stabilizing Factor VII Polypeptide Formulations

(75) Inventors: Anders Klarskov Petersen, Naerum (DK); Andrew Neil Bowler, Gentofte (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: NOVO NORDISK HEALTH CARE AG, Zurich (CH)

(21) Appl. No.: 11/817,029

(22) PCT Filed: Feb. 24, 2006
(86) PCT No.: PCT/EP06/60271

§ 371 Date: Oct. 3, 2008

(30) Foreign Application Priority Data
Feb. 24, 2005 (DK) PA 2005 00286

(51) Int. Cl. A61K 38/48; C07D 333/36; A61K 31/381; C12N 9/99; A61K 31/167; C07C 233/51

(52) U.S. Cl. 424/94.64; 549/69; 514/447; 514/616; 564/153; 435/183

(57) ABSTRACT

The invention relates to novel compounds with formula (I) and their use in stabilization of Factor VIIa or other Factor VII polypeptides, particularly in aqueous liquid compositions thereof.

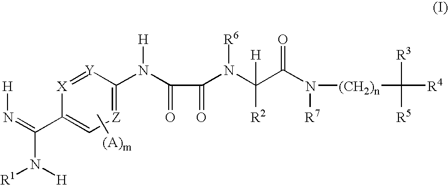

### FIELD OF THE INVENTION

The present invention relates to the field of stabilization of Factor VIIa or other Factor VII polypeptides (vide infra), particularly in aqueous liquid compositions thereof.

### BACKGROUND OF THE INVENTION

A variety of substances (“Factors”) involved in the blood clotting process have been identified, including Factor VII (FVII), which is a plasma glycoprotein. FVII exists in plasma mainly as a single-chain zymogen which is cleaved by another Factor, viz. Factor Xa (FXa), to give its two-chain, activated form, denoted Factor VIIa (FVIIa). Coagulation is initiated by the formation of a complex between FVIIa, which is present in the circulation in an amount corresponding to about 1% of the total FVII protein mass, and Tissue Factor (TF) which becomes exposed to the circulating blood following an injury to the vessel wall. Recombinant activated Factor VIIa (rFVIIa) has been developed as a pro-haemostatic agent.

It is desirable to have administration forms of Factor VIIa suitable for both storage and for delivery. Ideally, the drug product is stored and administered as a liquid. Alternatively, the drug product is lyophilized, i.e. freeze-dried, and then reconstituted by adding a suitable diluent prior to patient use. It is desirable that the drug product has sufficient stability to enable long-term storage, e.g. for more than six months.

The decision whether to maintain the finished drug product as a liquid or to freeze-dry it is usually made on the basis of the stability of the protein drug in those forms. Protein stability can be affected, inter alia, by such factors as ionic strength, pH, temperature, repeated cycles of freeze/thaw, and exposure to shear forces. Active protein may be lost as a result of physical instabilities, e.g. via denaturation and/or aggregation (both soluble and insoluble aggregate formation), as well as chemical instabilities, including, for example, instability towards hydrolysis, deamidation and/or oxidation, to name just a few. Moreover, in the case of Factor VIIa, which is a serine protease, fragmentation due to autocatalysis may occur (enzymatic degradation). For a general review of the stability of protein pharmaceuticals, see, for example, Manning, et al., Pharmaceutical Research 6:903-918 (1989).

While the possible occurrence of protein instabilities is widely appreciated, it is generally very difficult to predict particular instability problems for a particular protein. Any of these instabilities can result in the formation of a protein by-product, or derivative, having reduced activity, increased toxicity and/or increased immunogenicity.

Thus, the safety and efficacy of any composition of a protein is directly related to its stability. Maintaining stability in a liquid form is generally a different task than maintaining stability in a lyophilized form because of highly increased potential for molecular motion and thereby increased probability of molecular interactions. Maintaining stability in a concentrated form is also a different task than the above, because of the propensity for aggregate formation at increased protein concentrations. Factor VIIa undergoes degradation by several pathways, especially aggregation (dimerisation/oligomerisation), oxidation and autolytic cleavage (clipping of the peptide backbone or “heavy chain degradation”). Furthermore, precipitation may occur.

Many of these processes can be slowed significantly by removal of water from the protein. However, the development of an aqueous composition for Factor VIIa has the advantages of eliminating reconstitution errors, thereby increasing dosing accuracy, as well as simplifying the use of the product clinically, thereby increasing patient compliance. Ideally, compositions of Factor VIIa should be stable for more than 6 months over a wide range of protein concentrations. This allows for flexibility in methods of administration. Generally, more highly concentrated forms allow for the administration of lower volumes, which is highly desirable from the patients' point of view. Liquid compositions can have many advantages over freeze-dried products with regard to ease of administration and use.

Today, the only commercially available, recombinantly produced FVII polypeptide composition is a freeze-dried Factor FVIIa product which is reconstituted before use; it contains a relatively low Factor VIIa concentration, e.g., about 0.6 mg/mL. A vial (1.2 mg) of NovoSeven® (Novo Nordisk A/S, Denmark) contains 1.2 mg recombinant human Factor VIIa (rhFVIIA), 5.84 mg NaCl, 2.94 mg CaCl₂.2H₂O, 2.64 mg glycylglycine (GlyGly), 0.14 mg polysorbate 80, and 60.0 mg mannitol; it is reconstituted to pH 5.5 by addition of 2.0 mL water for injection (WFI). When reconstituted, the protein solution is stable for use for 24 hours at room temperature. Thus, no liquid, ready-for-use or concentrated Factor VII products are currently commercially available.

Thus, it is highly desirable to develop agents that inhibit degradation of FVII polypeptides in liquid (particularly aqueous liquid) or solid administration forms. It is particularly desirable to be able to provide an aqueous liquid pharmaceutical composition of a Factor VII polypeptide which provides acceptable control of chemical and/or physical degradation processes such as those outlined above.

### SUMMARY OF THE INVENTION

The present invention thus provides compounds of the formula I, below:

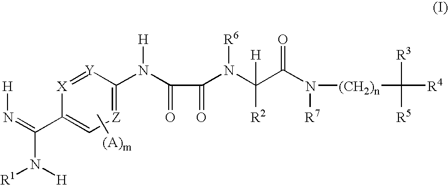

wherein R¹, R², R³, R⁴, R⁵, R⁶, R⁷, A, X, Y, Z, m and n have the meanings indicated below.

Compounds of formula I are capable of reversibly inhibiting the blood-clotting enzyme Factor VIIa (FVIIa), and may be employed as stabilizers in formulations or compositions, notably aqueous formulations or compositions, comprising FVIIa or another Factor VII polypeptide (vide infra).

The present invention further relates, inter alia, to:

(i) processes for the preparation of compounds of formula I (vide infra);
(ii) pharmaceutical compositions—notably liquid, aqueous pharmaceutical compositions comprising a Factor VII polypeptide (such as wild-type human FVIIa, e.g. rhFVIIa, or a variant or derivative thereof) and a compound of formula I; and
(iii) the use of such a pharmaceutical composition in the treatment of a condition or disorder against which the Factor VII polypeptide in question (such as wild-type human FVIIa, e.g. rhFVIIa, or a variant or derivative thereof) is effective;

### DETAILED DESCRIPTION OF THE INVENTION

As already indicated to some extent above, one aspect of the present invention relates to compounds of the formula I:

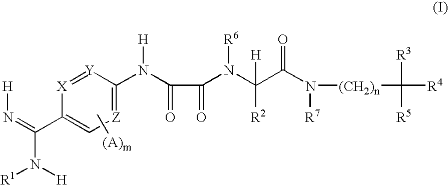

wherein
m is 0, 1 or 2;
n is 0 or 1;
A is halogen or hydroxy;
X, Y and Z independently are carbon or nitrogen;
or
X and Y taken together (i.e. the moiety X═Y) is a sulfur atom, and Z is carbon or nitrogen;
R¹ is chosen from hydrogen, hydroxy, (C₁-C₁₂)-alkoxycarbonyl-, (C₆-C₁₄)-aryl-(C₁-C₄)-alkoxycarbonyl-, and (C₆-C₁₄)-aryloxycarbonyl-, wherein each of the aryl groups is unsubstituted or substituted by at least one identical or different substituent chosen from (C₁-C₁₂)-alkyl, halogen, and (C₁-C₁₂)-alkoxy;
R² is chosen from hydrogen, (C₁-C₁₂)-alkyl, (C₆-C₁₄)-aryl, (C₆-C₁₄)-aryl-(C₁-C₄)-alkyl-, R²⁰—(C₁-C₁₂)-alkyl-, R², —(C₆-C₁₄)-aryl-, and R², —(C₆-C₁₄)-aryl-(C₁-C₄)-alkyl-, wherein R²⁰ is chosen from hydroxycarbonyl-, aminocarbonyl-, (C₁-C₁₂)-alkoxycarbonyl-, and (C₆-C₁₄)-aryl-(C₁-C₄)-alkoxycarbonyl-;
R³ is chosen from hydrogen, cyano, hydroxy, and (C₁-C₁₂)-alkyl;
R⁴ is chosen from (C₁-C₁₂)-alkyl, (C₆-C₁₄)-aryl, (C₆-C₁₄)-aryl-(C₁-C₄)-alkyl-, Het, and Het-(C₁-C₄)-alkyl-, wherein the alkyl, aryl and Het groups are unsubstituted or substituted by at least one identical or different substituent R¹⁰;
R⁵ is chosen from hydrogen, (C₁-C₁₂)-alkyl, (C₆-C₁₄)-aryl, (C₆-C₁₄)-aryl-(C₁-C₄)-alkyl-, Het, Het-(C₁-C₄)-alkyl-, (C₆-C₁₄)-aryl-(C₁-C₄)-alkyl-aminocarbonyl-, and Het-(C₁-C₄)-alkyl-aminocarbonyl-, wherein the alkyl, aryl and Het groups are unsubstituted or substituted by at least one identical or different substituent R¹⁰;
R⁶ and R⁷ independently are chosen from hydrogen and (C₁-C₈)-alkyl;
R¹⁰ is chosen from (C₁-C₁₂)-alkyl, (C₆-C₁₄)-aryl-(C₁-C₄)-alkyl-, (C₁-C₈)-alkoxy, (C₁-C₄)-alkoxy-(C₂-C₄)-alkoxy-, (C₆-C₁₄)-aryl-(C₁-C₄)-alkoxy-, (C₆-C₁₄)-aryloxy-, Het-oxy-, Het-(C₁-C₄)-alkoxy-, (C₆-C₁₄)-aryl, Het, Het-(C₁-C₄)-alkyl-, trifluoromethoxy, trifluoromethyl, halogen, oxo, hydroxy, amino, (C₁-C₁₂)-alkylcarbonylamino-, aminocarbonylamino-, (C₆-C₁₄)-arylcarbonylamino-, Het-carbonylamino-, (C₆-C₁₄)-aryl-(C₁-C₄)-alkylcarbonylamino-, Het-(C₁-C₄)-alkylcarbonylamino-, (C₁-C₈)-alkylcarbonyl-, (C₆-C₁₄)-arylcarbonyl-, (C₁-C₈)-alkylaminocarbonyl-, (C₆-C₁₄)-arylaminocarbonyl-, (C₆-C₁₄)-aryl-(C₁-C₄)-alkylaminocarbonyl-, Het-aminocarbonyl-, Het-(C₁-C₄)-alkylaminocarbonyl-, aminocarbonyl-, (C₁-C₈)-alkoxycarbonyl-, hydroxycarbonyl-, cyano, nitro, amidino, acetimino, tri-((C₁-C₄)-alkyl)ammonio-, (C₁-C₈)-alkylamino-, di-((C₁-C₈)-alkyl)amino-, hydroxycarbonylmethoxy-, (C₁-C₈)-alkylsulfonyl-, (C₆-C₁₄)-arylsulfonyl-, (C₁-C₈)-alkylaminosulfonyl-, (C₆-C₁₄)-arylaminosulfonyl-, (C₆-C₁₄)-aryl-(C₁-C₄)-alkylaminosulfonyl-, Het-aminosulfonyl-, Het-(C₁-C₄)-alkylaminosulfonyl-, (C₁-C₈)-alkylsulfonylamino-, (C₆-C₁₄)-arylsulfonylamino-, (C₆-C₁₄)-aryl-(C₁-C₄)-alkylsulfonylamino-, Het-sulfonylamino-, and Het-(C₁-C₄)-alkylsulfonylamino-, wherein (C₁-C₁₂)-alkylcarbonylamino-representing R¹⁰ is unsubstituted or substituted in the alkyl group by a substituent chosen from amino, hydroxy and (C₁-C₄)-alkoxy, and wherein (C₁-C₁₂)-alkyl and (C₁-C₈)-alkoxy representing R¹⁰ are unsubstituted or substituted by at least one identical or different substituent chosen from (C₁-C₈)-alkoxycarbonyl-, hydroxycarbonyl-, and aminocarbonyl-,
wherein each of the aryl groups and Het group in a group R¹⁰ is unsubstituted or substituted by at least one identical or different substituent chosen from halogen, nitro, oxo, hydroxy, (C₁-C₈)-alkyl, (C₁-C₈)-alkoxy, (C₁-C₄)-alkoxy-(C₂-C₄)-alkoxy-, (C₆-C₁₄)-aryloxy-, (C₆-C₁₄)-aryl-(C₁-C₄)-alkoxy-, Het-oxy-, Het-(C₁-C₄)-alkoxy-, (C₆-C₁₄)-aryl, (C₆-C₁₄)-aryl(C₁-C₄)-alkyl-, Het, Het-(C₁-C₄)-alkyl-, trifluoromethyl, cyano, trifluoromethoxy, (C₁-C₈)-alkylsulfonyl-, (C₁-C₈)-alkoxycarbonyl-, hydroxycarbonyl-, aminocarbonyl-, amino, (C₁-C₈)-alkylamino-, di-((C₁-C₈)-alkyl)amino-, (C₁-C₈)-alkylcarbonylamino-, (C₆-C₁₄)-aryl-(C₁-C₄)-alkylcarbonylamino-, (C₆-C₁₄)-arylcarbonylamino-, Het-carbonylamino-, Het-(C₁-C₄)-alkylcarbonylamino-, and (C₁-C₈)-alkylcarbonyl-, wherein (C₁-C₈)-alkyl and (C₁-C₈)-alkoxy representing a substituent on an aryl group or Het group in a group R¹¹ are un-substituted or substituted by at least one identical or different substituent chosen from (C₁-C₈)-alkoxycarbonyl-, hydroxycarbonyl-, and aminocarbonyl-;
Het is a residue of a saturated or unsaturated monocyclic or bicyclic, 3-membered to 10-membered heterocyclic ring system containing 1, 2 or 3 identical or different ring heteroatoms chosen from nitrogen, oxygen and sulfur;
including any and all stereoisomeric form or forms thereof;
and any mixture of two or more such compounds of formula I in any ratio;
and physiologically tolerable salts thereof.

All groups, substituents, residues etc. which can occur several times in the compounds of the formula I, for example A, R¹⁰ or Het, can each independently of one another have the meanings indicated, and can in each case be identical or different.

As used herein, the term alkyl is to be understood in the broadest sense to mean hydrocarbon residues which can be linear, i.e. straight-chain, or branched and which can be acyclic or cyclic groups or comprise any combination of acyclic and cyclic subunits. Further, the term alkyl as used herein expressly includes saturated groups as well as unsaturated groups which latter groups contain one or more, for example one, two or three, double bonds and/or triple bonds, provided that the double bonds are not located within a cyclic alkyl group in such a manner that an aromatic system results. All these statements also apply if an alkyl group occurs as a substituent on another group or is substituted, for example in an alkoxy group (alkyl-O—), an alkoxycarbonyl- group or an arylalkyl- group. Non limiting examples of alkyl groups containing 1, 2, 3, 4, 5, 6, 7, 8, 9, 10, 11, or 12 carbon atoms are methyl, ethyl, propyl, butyl, pentyl, hexyl, heptyl, octyl, nonyl, decyl, undecyl, dodecyl, the n-isomers of all these groups, isopropyl, isobutyl, 1-methylbutyl, isopentyl, neopentyl, 2,2-dimethylbutyl, 2-methylpentyl, 3-methylpentyl, isohexyl, sec-butyl, tert-butyl, tert-pentyl, 2,3,4-trimethylhexyl, and isodecyl.

Unsaturated alkyl groups are, for example, alkenyl groups such as vinyl, 1-propenyl, 2-propenyl, (=allyl), 2-butenyl, 3-butenyl, 2-methyl-2-butenyl, 3-methyl-2-butenyl, 5-hexenyl, or 1,3-pentadienyl, or alkynyl groups such as ethynyl, 1-propynyl, 2-propynyl (=propargyl), or 2-butynyl. Alkyl groups can also be unsaturated when they are substituted.

Non-limiting examples of cyclic alkyl groups are cycloalkyl groups containing 3, 4, 5, 6, 7, or 8 ring carbon atoms like cyclopropyl, cyclobutyl, cyclopentyl, cyclohexyl, cycloheptyl, or cyclooctyl, which can also be substituted and/or unsaturated. Unsaturated cyclic alkyl groups and unsaturated cycloalkyl groups such as, for example, cyclopentenyl or cyclohexenyl, can be bonded via any carbon atom. The term alkyl as used herein also comprises cycloalkyl-substituted alkyl groups such as cyclopropylmethyl-, cyclobutylmethyl-, cyclopentylmethyl-, cyclohexylmethyl-, cycloheptylmethyl-, 1-cyclopropylethyl-, 1-cyclobutylethyl-, 1-cyclopentylethyl-, 1-cyclohexylethyl-, 2-cyclopropylethyl-, 2-cyclobutylethyl-, 2-cyclopentylethyl-, 2-cyclohexylethyl-, 3-cyclopropylpropyl-, 3-cyclobutylpropyl-, 3-cyclopentylpropyl-, and others, in which groups the cycloalkyl sub-group as well as acyclic subgroup can be unsaturated and/or substituted.

Of course, a cyclic alkyl group has to contain at least three carbon atoms, and an unsaturated alkyl group has to contain at least two carbon atoms. Thus, a group like (C₁-C₈)-alkyl is to be understood as comprising, among others, saturated acyclic (C₁-C₈)-alkyl, (C₃-C₈)-cycloalkyl, cycloalkyl-alkyl-groups like (C₃-C₇)-cycloalkyl-(C₁-C₃)-alkyl- wherein the total number of carbon atoms can range from 4 to 8, and unsaturated (C₂-C₈)-alkyl like (C₂-C₈)-alkenyl or (C₂-C₈)-alkynyl. Similarly, a group like (C₁-C₄)-alkyl is to be understood as comprising, among others, saturated acyclic (C₁-C₄)-alkyl, (C₃-C₄)-cycloalkyl, cyclopropyl-methyl- and unsaturated (C₂-C₄)-alkyl like (C₂-C₄)-alkenyl or (C₂-C₄)-alkynyl.

Unless stated otherwise, in one embodiment of the invention the term alkyl comprises acyclic saturated hydrocarbon residues which have from one to six carbon atoms and which can be linear or branched. A particular group of saturated acyclic alkyl groups is formed by (C₁-C₄)-alkyl groups such as methyl, ethyl, n-propyl, isopropyl, n-butyl, isobutyl, sec-butyl or tert-butyl.

The above statements relating to alkyl groups do not only apply to monovalent residues, but correspondingly to divalent and polyvalent residues such as alkanediyl groups, alkylene groups or polymethylene groups, some examples of which are methylene, 1,2-ethylene (=ethane-1,2-diyl), 1,1-ethylene (=1-methyl-methylene), 1-isobutylmethylene, 1,3-propylene, 2,2-dimethyl-1,3-propylene, 1,4-butylene, but-2-en-1,4-diyl, 1,2-cyclopropylene, 1,2-cyclohexylene, 1,3-cyclohexylene, and 1,4-cyclohexylene.

Examples of (C₁-C₄)-alkoxy-(C₂-C₄)-alkoxy-groups are 2-methoxyethoxy-, 2-ethoxyethoxy-, 2-isopropoxyethoxy-, 3-methoxypropoxy-, and 4-ethoxybutoxy-.

The term aryl refers to a monocyclic or polycyclic hydrocarbon residue in which residue at least one carbocyclic ring is present which has a conjugated pi electron system, i.e., which is an aromatic ring, and which residue is attached via a carbon atom contained in a ring which has a conjugated pi electron system. In a (C₆-C₁₄)-aryl group from 6 to 14 ring carbon atoms are present. Examples of (C₆-C₁₄)-aryl groups are phenyl, naphthyl, biphenylyl, fluorenyl, anthracenyl, indenyl, indanyl, 1,2,3,4-tetrahydronaphthyl or 2,3,4,5-tetrahydro-1H-benzocycloheptenyl. Examples of (C₆-C₁₀)-aryl groups are phenyl, naphthyl, indenyl, indanyl or 1,2,3,4-tetrahydronaphthyl. Unless stated otherwise, and irrespective of any specific substituents bonded to aryl groups which are indicated in the definition of the compounds of the formula I, aryl groups, for example phenyl, naphthyl or fluorenyl, can in general be unsubstituted or substituted by one or more, for example one, two, three, or four, identical or different substituents, for example by the substituents listed below.

Aryl groups can be bonded via any desired position in an aromatic ring. In substituted aryl groups the substituents can be located in any desired position. In monosubstituted phenyl groups the substituent can be located in the 2-position, the 3-position or the 4-position, such as in the 3-position or the 4-position. If a phenyl group carries two substituents, they can be located in 2,3-position, 2,4-position, 2,5-position, 2,6-position, 3,4-position or 3,5-position. In phenyl groups carrying three substituents the substituents can be located in 2,3,4-position, 2,3,5-position, 2,3,6-position, 2,4,5-position, 2,4,6-position, or 3,4,5-position. Naphthyl groups can be 1-naphthyl (=naphthalen-1-yl) and 2-naphthyl (=naphthalen-2-yl). In substituted naphthyl groups the substituents can be located in any positions, for example in monosubstituted 1-naphthyl groups in the 2-, 3-, 4-, 5-, 6-, 7-, or 8-position and in monosubstituted 2-naphthyl groups in the 1-, 3-, 4-, 5-, 6-, 7-, or 8-position. 1,2,3,4-Tetrahydronaphthyl, when attached via a carbon atom in the aromatic ring and comprised by the term aryl, can be 1,2,3,4-tetrahydronaphthalen-5-yl or 1,2,3,4-tetrahydronaphthalen-6-yl. Biphenylyl groups can be biphenyl-2-yl, biphenyl-3-yl or biphenyl-4-yl. Fluorenyl groups, when comprised by the term aryl, can be bonded via the 1-, 2-, 3-, or 4-position, otherwise via the 1-, 2-, 3-, 4- or 9-position. In one embodiment of the invention, in monosubstituted fluorenyl groups bonded via the 9-position, the substituent can be present in the 1-, 2-, 3- or 4-position.

The above statements relating to aryl groups correspondingly apply to divalent and polyvalent groups derived from aryl groups, e.g. to arylene groups like phenylene which can be unsubstituted or substituted 1,2-phenylene, 1,3-phenylene or 1,4-phenylene, or naphthylene which can be unsubstituted or substituted 1,2-naphthalenediyl, 1,3-naphthalenediyl, 1,4-naphthalenediyl, 1,5-naphthalenediyl, 1,6-naphthalenediyl, 1,7-naphthalenediyl, 1,8-naphthalenediyl, 2,3-naphthalenediyl, 2,6-naphthalenediyl, or 2,7-naphthalenediyl. The above statements also correspondingly apply to the aryl sub-group in arylalkyl-groups. Examples of arylalkyl-groups, which can also be unsubstituted or substituted in the aryl subgroup as well as in the alkyl subgroup, are benzyl, 1-phenylethyl, 2-phenylethyl, 1-phenylpropyl, 2-phenylpropyl, 3-phenylpropyl, 1-phenylbutyl, 4-phenylbutyl, 1-methyl-3-phenyl-propyl, 1-naphthylmethyl, 2-naphthylmethyl, 1-(1-naphthyl)ethyl, 1-(2-naphthyl)ethyl, 2-(1-naphthyl)ethyl, 2-(2-naphthyl)ethyl, and 9-fluorenylmethyl. All the above explanations also apply to aromatic rings which may be condensed (or fused) to a ring formed by the groups R⁴ and R⁵ and the carbon atom to which these groups are attached.

The Het group comprises groups containing 3, 4, 5, 6, 7, 8, 9, or 10 ring atoms in the parent monocyclic or bicyclic heterocyclic ring system. In monocyclic Het groups the heterocyclic ring may comprise a 3-membered, 4-membered, 5-membered, 6-membered or 7-membered ring, such as a 5-membered or 6-membered ring. In bicyclic Het groups two fused rings may be present, one of which is a 5-membered ring or 6-membered heterocyclic ring and the other of which is a 5-membered or 6-membered heterocyclic or carbocyclic ring. For example, a bicyclic ring Het may contain 8, 9 or 10 ring atoms, for example, 9 or 10 ring atoms.

Het comprises saturated heterocyclic ring systems which do not contain any double bonds within the rings, as well as unsaturated heterocyclic ring systems including monounsaturated and poly-unsaturated heterocyclic ring systems which contain one or more, for example one, two, three, four, or five, double bonds within the rings provided that the resulting system is stable. Unsaturated rings may be partially unsaturated or nonaromatic, or they may be aromatic and thus may contain double bonds arranged in such a manner that a conjugated pi electron system results. Aromatic rings in a Het group may be 5-membered or 6-membered rings. For example, aromatic groups in a Het group contain 5 to 10 ring atoms. Aromatic rings in a Het group thus comprise 5-membered and 6-membered monocyclic heterocycles and bicyclic heterocycles composed of two 5-membered rings, one 5-membered ring and one 6-membered ring, or two 6-membered rings. In bicyclic aromatic groups in a Het group one or both rings may contain heteroatoms. Aromatic Het groups may also be referred to by the customary term heteroaryl for which all the definitions and explanations above and below relating to Het correspondingly apply. These explanations relating to the saturation/unsaturation in heterocyclic ring systems representing the Het group corresponding apply to any other heterocyclic ring system that can be present in a compound of the formula I, for example to a ring formed by R⁴ and R⁵ together with the carbon atom to which these groups are bonded, and the ring systems that may be condensed to this ring.

In a Het group and any other heterocyclic group, for example, 1 or 2 identical or different ring heteroatoms selected from nitrogen, oxygen and sulfur atoms may be present. In general, the ring heteroatoms can be present in any desired combination and in any desired positions with respect to each other provided that the resulting heterocyclic system is known in the art and is stable and suitable as a subgroup in a drug substance. Examples of parent structures of heterocycles from which the Het group any other heterocyclic groups can be derived are aziridine, oxirane, azetidine, pyrrole, furan, thiophene, dioxole, imidazole, pyrazole, oxazole, isoxazole, thiazole, isothiazole, 1,2,3-triazole, 1,2,4-triazole, pyridine, pyran, thiopyran, pyridazine, pyrimidine, pyrazine, 1,4-dioxine, 1,2-oxazine, 1,3-oxazine, 1,4-oxazine, 1,2-thiazine, 1,3-thiazine, 1,4-thiazine, 1,2,3-triazine, 1,2,4-triazine, 1,3,5-triazine, azepine, 1,2-diazepine, 1,3-diazepine, 1,4-diazepine, indole, isoindole, benzofuran, benzothiophene, 1,3-benzodioxole, benzo[1,4]dioxine, 4H-benzo[1,4]oxazine, indazole, benzimidazole, benzoxazole, benzothiazole, quinoline, isoquinoline, chromane, isochromane, cinnoline, quinazoline, quinoxaline, phthalazine, pyridoimidazoles, pyridopyridines, and pyridopyrimidines, and others, as well as ring systems which result from the listed heterocycles by fusion (or condensation) of a carbocyclic ring, for example benzo-fused, cyclopenta-fused, cyclohexa-fused or cyclohepta-fused derivatives of these heterocycles.

The fact that many of the before-listed names of heterocycles are the chemical names of unsaturated or aromatic ring systems does not imply that the Het groups and other heterocyclic groups could only be derived from the respective unsaturated ring system. The names here only serve to describe the ring system with respect to ring size and the number of the heteroatoms and their relative positions. As explained above, for example a Het group can be saturated or partially unsaturated or aromatic, and can thus be derived not only from the before-listed heterocycles themselves but also from all of their partially or completely hydrogenated analogues as well as from their more highly unsaturated analogues, if applicable. As examples of completely or partially hydrogenated analogues of the before-listed heterocycles from which a Het group and any other heterocyclic group may be derived the following may be mentioned: pyrroline, pyrrolidine, tetrahydrofuran, tetrahydrothiophene, dihydropyridine, tetrahydropyridine, piperidine, 1,3-dioxolane, 2-imidazoline, imidazolidine, 4,5-dihydro-1,3-oxazole, 1,3-oxazolidine, 4,5-dihydro-1,3-thiazole, 1,3-thiazolidine, perhydro-1,4-dioxine (=1,4-dioxane), piperazine, perhydro-1,4-oxazine (=morpholine), 2,3-dihydrobenzo[1,4]dioxine (=1,4-benzodioxane), 3,4-dihydro-2H-benzo[1,4]oxazine, perhydro-1,4-thiazine (=thiomorpholine), perhydroazepine, indoline, isoindoline, 1,2,3,4-tetrahydroquinoline, and 1,2,3,4-tetrahydroisoquinoline, and others.

The Het group and any other heterocyclic group may, unless otherwise indicated, be bonded via any ring carbon atom, and in the case of nitrogen heterocycles via any suitable ring nitrogen atom, if applicable. Thus, for example, a pyrrolyl group can be pyrrol-1-yl, pyrrol-2-yl or pyrrol-3-yl, a pyrrolidinyl group can be pyrrolidin-1-yl (=pyrrolidino), pyrrolidin-2-yl or pyrrolidin-3-yl, a pyridinyl group can be pyridin-2-yl, pyridin-3-yl or pyridin-4-yl, a piperidinyl group can be piperidin-1-yl (=piperidino), piperidin-2-yl, piperidin-3-yl or piperidin-3-yl. Furyl can be furan-2-yl or fur-3-yl, thienyl can be thiophen-2-yl or thiophen-3-yl, imidazolyl can be imidazol-1-yl, imidazol-2-yl, imidazol-4-yl, or imidazol-5-yl, 1,3-oxazolyl can be 1,3-oxazol-2-yl, 1,3-oxazol-4-yl or 1,3-oxazol-5-yl, 1,3-thiazolyl can be 1,3-thiazol-2-yl, 1,3-thiazol-4-yl, or 1,3-thiazol-5-yl, pyrimidinyl can be pyrimidin-2-yl, pyrimidin-4-yl (=pyrimidin-6-yl) or pyrimidin-5-yl, piperazinyl can be piperazin-1-yl (=piperazin-4-yl=piperazino) or piperazin-2-yl. Indolyl can be indol-1-yl, indol-2-yl, indol-3-yl, indol-4-yl, indol-5-yl, indol-6-yl, or indol-7-yl. Similarly benzimidazolyl, benzoxazolyl and benzothiazolyl groups can be bonded via the 2-position and via any of the positions 4, 5, 6, and 7, and in the case of benzimidazolyl, also via the 1-position. Quinolinyl can be quinolin-2-yl, quinolin-3-yl, quinolin-4-yl, quinolin-5-yl, quinolin-6-yl, quinolin-7-yl, or quinolin-8-yl, isoqinolinyl can be isoquinolin-1-yl, isoquinolin-3-yl, isoquinolin-4-yl, isoquinolin-5-yl, isoquinolin-6-yl, isoquinolin-7-yl, or isoquinolin-8-yl. In addition to being bonded via any of the positions indicated for quinolinyl and isoquinolinyl, 1,2,3,4-tetrahydroquinolinyl and 1,2,3,4-tetrahydroisoquinolinyl can also be bonded via the nitrogen atoms in 1-position and 2-position, respectively.

Unless stated otherwise, and irrespective of any specific substituents in aryl groups, Het groups or any other heterocyclic groups which are indicated in the definition of the compounds of the formula I, aryl groups, Het groups and other heterocyclic groups can be unsubstituted or substituted on ring carbon atoms with one or more, for example one, two, three, four or five, identical or different substituents like (C₁-C₄)-alkyl, (C₁-C₄)-alkoxy, (C₁-C₄)-alkylthio, halogen, nitro, amino, (C₁-C₄)-alkylamino, di-((C₁-C₄)-alkyl)amino, ((C₁-C₄)-alkyl)-carbonylamino such as, for example, acetylamino, trifluoromethyl, trifluoromethoxy, hydroxy, oxo, hydroxymethyl, methylenedioxy, ethylenedioxy, formyl, acetyl, cyano, methylsulfonyl, optionally substituted phenyl, optionally substituted phenoxy, benzyl optionally substituted in the phenyl group, benzyloxy optionally substituted in the phenyl group, and others. The substituents can be present in any desired position provided that a stable molecule results. An oxo substituent (═O) can of course not be present in an aromatic ring, but can be present if the Het group or any other heterocyclic or carbocyclic group is saturated or partially unsaturated. Examples of oxo-substituted heterocyclic groups are 4H-benzo[1,4]oxazin-3-one, 3H-isobenzofuran-1-one, benzo[1,4]dioxin-2-one, chroman-2-one, and others. Examples of the group Het-oxy-, i.e. the group Het-O—, are pyridinyloxy including pyridin-3-yloxy and pyridin-4-yloxy, pyrimidinyloxy including pyrimidin-2-yloxy, piperidinyloxy including piperidin-3-yloxy and piperidin-4-yloxy, and pyrrolidin-3-yloxy. In some embodiments of the invention, not more than two nitro groups are present in the compounds of the formula I.

Further, unless stated otherwise, and irrespective of any specific substituents in Het groups or any other heterocyclic groups which are indicated in the definition of the compounds of the formula I, Het groups and other heterocyclic groups can on each suitable ring nitrogen atom independently of one another be unsubstituted, i.e. carry a hydrogen atom, or be substituted, for example, by (C₁-C₈)-alkyl, for example (C₁-C₄)-alkyl such as methyl or ethyl, optionally substituted phenyl, phenyl-(C₁-C₄)-alkyl-, for example benzyl, optionally substituted in the phenyl group, hydroxy-(C₂-C₄)-alkyl- such as, for example 2-hydroxyethyl, acetyl or another acyl group, methylsulfonyl or another sulfonyl group, etc. Another group that may occur as a substituent on a suitable ring nitrogen atom is the acetimino group CH₃—C(═NH)—. Suitable nitrogen heterocycles can also be present as N-oxides or as quaternary salts. Ring sulfur atoms can be oxidized to the sulfoxide or to the sulfone. Thus, for example, a tetrahydrothienyl residue may be present as S,S-dioxotetrahydrothienyl residue or a thiomorpholinyl residue like thiomorpholin-4-yl may be present as 1-oxo-thiomorpholin-4-yl or 1,1-dioxo-thiomorpholin-4-yl.

The explanations relating to the Het group correspondingly apply to divalent and polyvalent Het groups including divalent and polyvalent heteroaromatic groups which may be bonded via any ring carbon atoms and in the case of nitrogen heterocycles via any carbon atoms and any suitable ring nitrogen atoms or via any suitable ring nitrogen atoms. For example, a pyridinediyl group can be pyridin-2,3-diyl, pyridin-2,4-diyl, pyridin-2,5-diyl, pyridin-2,6-diyl, pyridin-3,4-diyl, or pyridin-3,5-diyl, a piperidinediyl group can be, among others, piperidin-1,2-diyl, piperidin-1,3-diyl, piperidin-1,4-diyl, piperidin-2,3-diyl, piperidin-2,4-diyl, or piperidin-3,5-diyl, a piperazinediyl group can be, among others, piperazin-1,3-diyl, piperazin-1,4-diyl, piperazin-2,3-diyl, piperazin-2,5-diyl, and others. The above statements also correspondingly apply to the Het subgroup in the groups Het-alkyl-. Examples of such groups Het-alkyl- which can also be unsubstituted or substituted in the Het subgroup as well as in the alkyl subgroup, are (pyridin-2-yl)-methyl, (pyridin-3-yl)-methyl, (pyridin-4-yl)-methyl, 2-(pyridin-2-yl)-ethyl, 2-(pyridin-3-yl)-ethyl, and 2-(pyridin-4-yl)-ethyl.

Halogen is fluorine, chlorine, bromine or iodine, in some embodiments being fluorine, chlorine or bromine, such as fluorine or chlorine.

Stereogenic carbon atoms present in the compounds of the formula I can independently of each other have R configuration or S configuration. The compounds of the formula I can be present in the form of pure enantiomers or pure diastereomers or in the form of mixtures of enantiomers and/or diastereomers, for example in the form of racemates. The present invention relates to pure enantiomers and mixtures of enantiomers as well as to pure diastereomers and mixtures of diastereomers. The invention comprises mixtures of two or of more than two stereoisomers of the formula I, and it comprises all ratios of the stereoisomers in the mixtures. In case the compounds of the formula I can be present as E isomers or Z isomers (or cis isomers or trans isomers) the invention relates both to pure E isomers and pure Z isomers and to E/Z mixtures in all ratios. The invention also comprises all tautomeric forms of the compounds of the formula I, for example the form in which instead of the group R¹—NH—C(═NH)— depicted in formula I the tautomeric group R¹—N(═C)(—NH₂)— is present.

Diastereomers, including E/Z isomers, can be separated into the individual isomers, for example, by chromatography. Racemates can be separated into the two enantiomers by customary methods, for example by chromatography on chiral phases or by resolution, for example by crystallization of diastereomeric salts obtained with optically active acids or bases. Stereochemically uniform compounds of the formula I can also be obtained by employing stereochemically uniform starting materials or by using stereoselective reactions.

The choice of incorporating into a compound of the formula I a building block with R configuration or S configuration, or in the case of an amino acid unit present in a compound of the formula I of incorporating a building block designated as D-amino acid or L-amino acid, can depend, for example, on the desired characteristics of the compound of the formula I. For example, the incorporation of a D-amino acid building block can confer increased stability in vitro or in vivo. The incorporation of a D-amino acid building block also can achieve a desired increase or decrease in the pharmacological activity of the compound. In some cases it can be desirable to allow the compound to remain active for only a short period of time. In such cases, the incorporation of an L-amino acid building block into the compound can allow endogenous peptidases in an individual to digest the compound in vivo, thereby limiting the individual's exposure to the active compound. A similar effect may also be observed in the compounds of the invention by changing the configuration in another building block from S configuration to R configuration or vice versa. By taking into consideration the medical needs one skilled in the art can determine the desirable characteristics, for example a favorable stereochemistry, of the required compound of the invention.

Physiologically tolerable salts of the compounds of formula I are nontoxic salts that are physiologically acceptable, such as pharmaceutically utilizable salts. Such salts of compounds of the formula I containing acidic groups, for example a carboxy group COOH, are, for example, alkali metal salts or alkaline earth metal salts such as sodium salts, potassium salts, magnesium salts and calcium salts, and also salts with physiologically tolerable quaternary ammonium ions such as tetramethylammonium or tetraethylammonium, and acid addition salts with ammonia and physiologically tolerable organic amines, such as methylamine, dimethylamine, trimethylamine, ethylamine, triethylamine, ethanolamine or tris-(2-hydroxyethyl)amine. Basic groups contained in the compounds of the formula I, for example amino groups or amidino groups, form acid addition salts, for example with inorganic acids such as hydrochloric acid, hydrobromic acid, sulfuric acid, nitric acid or phosphoric acid, or with organic carboxylic acids and sulfonic acids such as formic acid, acetic acid, oxalic acid, citric acid, lactic acid, malic acid, succinic acid, malonic acid, benzoic acid, maleic acid, fumaric acid, tartaric acid, methanesulfonic acid or p-toluenesulfonic acid. The present invention also includes acid addition salts of compounds of the formula I which contain, for example, two basic groups with one acid equivalent or with two acid equivalents.

Salts of compounds of the formula I can be obtained by customary methods known to those skilled in the art, for example by combining a compound of the formula I with an inorganic or organic acid or base in a solvent or diluent, or from other salts by cation exchange or anion exchange. The present invention also includes all salts of the compounds of the formula I which, because of low physiological tolerability, are not directly suitable for use in pharmaceuticals but are suitable, for example, as intermediates for carrying out further chemical modifications of the compounds of the formula I or as starting materials for the preparation of physiologically tolerable salts.

The anions of the mentioned acids that may be present in acid addition salts of the compounds of the formula I, are also examples of anions that may be present in the compounds of the formula I if they contain one or more positively charged groups like trialkylammonio-substituents, i.e. groups of the formula (alkyl)₃N⁺ bonded via the positively charged nitrogen atom, which groups may represent R¹⁰, or quaternized ring nitrogen atoms in heterocyclic groups. In general a compound of the formula I contains one or more physiologically tolerable anions or anion equivalents as counterions if it contains one or more permanently positively charged groups like trialkylammonio. Compounds of the formula I which simultaneously contain a basic group or a positively charged group and an acidic group, for example an amidino group and a carboxy group, can also be present as zwitterions (or betaines or inner salts) which are likewise included in the present invention.

The present invention furthermore includes all solvates of compounds of the formula I, for example hydrates or adducts with alcohols.

The structural elements in the compounds of formula I have the following example denotations which they can have independently of the denotations of other elements.

The parameter m, i.e. the number of halogen atoms or hydroxy groups that are present as substituents on the aromatic/heteroaromatic group (containing X, Y and Z) depicted in formula I, can have the values 0, 1 or 2. In some embodiments of compounds of the invention, m may be 0 or 1, or 0. Those remaining carbon atoms in the aromatic/heteroaromatic group in question which do not carry a substituent A carry hydrogen atoms.

When m=1, the substituent A is, as already indicated, selected among halogens and hydroxy, e.g. hydroxy. When m=2, the substituents A can be identical or different, and are independently selected among halogens and hydroxy. In some embodiments of compounds of the invention, A may be selected from fluorine, chlorine and hydroxy.

The parameter n can have the values 0 or 1. In some embodiments of compounds of the invention, n may be 0.

In some embodiments of compounds of the invention, R¹ is selected from hydrogen, hydroxy and (C₁-C₁₂)-alkoxycarbonyl-. In certain embodiments, R¹ may suitably be hydrogen.

If an aryl group present in a group R¹ is substituted by one or more identical or different substituents selected from (C₁-C₁₂)-alkyl, halogen and (C₁-C₁₂)-alkoxy, it can be substituted by 1, 2 or 3, such as by 1 or 2, identical or different substituents, for example by one substituent. An alkyl group or alkoxy group present in a substituent in a group R¹ can be a (C₁-C₄)-alkyl group or (C₁-C₄)-alkoxy group, respectively.

In some embodiments of compounds of the invention, R² is selected from hydrogen, (C₁-C₁₂)-alkyl, (C₆-C₁₄)-aryl and (C₆-C₁₄)-aryl-(C₁-C₄)-alkyl-, such as from hydrogen, (C₁-C₈)-alkyl, (C₆-C₁₀)-aryl and (C₆-C₁₀)-aryl-(C₁-C₄)-alkyl-. In certain embodiments, R² may suitably be hydrogen.

In some embodiments of compounds of the invention, R³ may suitably be hydrogen.

In some embodiments of compounds of the invention, R⁴ is selected from (C₁-C₈)-alkyl, (C₆-C₁₀)-aryl, (C₆-C₁₀)-aryl-(C₁-C₄)-alkyl-, Het and Het-(C₁-C₄)-alkyl-, where the alkyl, aryl and Het groups are unsubstituted or substituted with one or more identical or different substituents R¹⁰. In certain embodiments, R⁴ is (C₆-C₁₀)-aryl or Het, such as (C₆-C₁₀)-aryl, for example phenyl, where the aryl, Het and phenyl groups are unsubstituted or substituted with one or more identical or different substituents R¹⁰.

In some embodiments of compounds of the invention, R⁵ is selected from hydrogen, (C₁-C₁₂)-alkyl, (C₆-C₁₄)-aryl, (C₆-C₁₄)-aryl-(C₁-C₄)-alkyl-, Het and Het-(C₁-C₄)-alkyl-, such as from hydrogen, (C₁-C₈)-alkyl, (C₆-C₁₀)-aryl, (C₆-C₁₀)-aryl-(C₁-C₄)-alkyl-, Het and Het-(C₁-C₄)-alkyl-, or such as from hydrogen, (C₁-C₈)-alkyl, (C₆-C₁₀)-aryl and (C₆-C₁₀)-aryl-(C₁-C₄)-alkyl-, or such as from hydrogen, (C₁-C₄)-alkyl and phenyl, or such as from (C₁-C₄)-alkyl and phenyl, for example from methyl, ethyl and phenyl, where the alkyl, aryl, phenyl and Het groups are unsubstituted or substituted with one or more identical or different substituents R¹⁰. In certain embodiments, R⁵ is (C₁-C₄)-alkyl, for example methyl or ethyl.

In some embodiments of compounds of the invention, R⁶ may suitably be hydrogen or (C₁-C₄)-alkyl, for example hydrogen.

In some embodiments of compounds of the invention, R⁷ may suitably be hydrogen or (C₁-C₄)-alkyl, for example hydrogen.

In some embodiments of compounds of the invention, an aryl group or Het group, such as an aryl group, representing R⁴ or R⁵ or present in the aryl or Het part of an arylalkyl- or Het-alkyl- group representing R⁴ or R⁵, for example a group representing R⁴, is substituted with one or more identical or different substituents R¹⁰. Within said embodiments, a subgroup of compounds is formed by compounds in which the substituent R¹⁰ or the substituents R¹⁰ on aryl groups or Het groups representing R⁴ or R⁵, or present in the aryl or Het part of an arylalkyl- or Het-alkyl- group representing R⁴ or R⁵, are selected from halogen (for example fluorine, chlorine and bromine, e.g. bromine), (C₁-C₈)-alkyl, (C₁-C₈)-alkoxy, (C₆-C₁₀)-aryloxy- and (C₆-C₁₀)-aryl-(C₁-C₄)-alkoxy-, where each of the aryl groups in a group R¹⁰ is unsubstituted or substituted as defined above. Another sub-group of compounds within said embodiments of compounds of the invention is formed by compounds in which aryl groups or Het groups representing R⁴ or R⁵, or present in the aryl or Het part of an arylalkyl- or Het-alkyl- group representing R⁴ or R⁵, are substituted with one substituent R¹⁰ which is selected from (C₁-C₈)-alkylcarbonylamino-, (C₆-C₁₀)-arylcarbonylamino-, (C₆-C₁₀)-aryl-(C₁-C₄)-alkylcarbonylamino-, Het-carbonylamino-, Het-(C₁-C₄)-alkylcarbonylamino-, (C₁-C₈)-alkylaminocarbonyl-, (C₆-C₁₀)-arylaminocarbonyl-, (C₆-C₁₀)-aryl-(C₁-C₄)-alkylaminocarbonyl-, Het-aminocarbonyl-, Het-(C₁-C₄)-alkylaminocarbonyl-, (C₁-C₈)-alkylaminosulfonyl-, (C₆-C₁₀)-arylaminosulfonyl-, (C₆-C₁₀)-aryl-(C₁-C₄)-alkylaminosulfonyl-, Het-aminosulfonyl-, Het-(C₁-C₄)-alkylaminosulfonyl-, (C₁-C₈)-alkylsulfonylamino-, (C₆-C₁₀)-arylsulfonylamino-, (C₆-C₁₀)-aryl-(C₁-C₄)-alkylsulfonylamino-, Het-sulfonylamino- and Het-(C₁-C₄)-alkylsulfonylamino-, and substituted with zero, one or two identical or different substituents R¹⁰ which are selected from halogen (for example fluorine, chlorine and bromine, e.g. bromine), (C₁-C₈)-alkyl, (C₁-C₈)-alkoxy, (C₆-C₁₀)-aryloxy- and (C₆-C₁₀)-aryl-(C₁-C₄)-alkoxy-, where each of the aryl groups and Het groups in a group R¹⁰ is unsubstituted or substituted as defined above. Further subgroups of compounds within said embodiments of compounds of the invention are formed by compounds in which aryl groups or Het groups representing R⁴ or R⁵, or present in the aryl or Het part of an arylalkyl- or Het-alkyl- group representing R⁴ or R⁵, are substituted with one substituent R¹⁰ which is selected from (C₁-C₈)-alkylcarbonylamino-, (C₆-C₁₀)-arylcarbonylamino-, (C₆-C₁₀)-aryl-(C₁-C₄)-alkylcarbonylamino-, Het-carbonylamino- and Het-(C₁-C₄)-alkylcarbonylamino-, or from (C₁-C₈)-alkylaminocarbonyl-, (C₆-C₁₀)-arylaminocarbonyl-, (C₆-C₁₀)-aryl-(C₁-C₄)-alkylaminocarbonyl-, Het-aminocarbonyl- and Het-(C₁-C₄)-alkylaminocarbonyl-, or from (C₁-C₈)-alkylaminosulfonyl-, (C₆-C₁₀)-arylaminosulfonyl-, (C₆-C₁₀)-aryl-(C₁-C₄)-alkylaminosulfonyl-, Het-aminosulfonyl- and Het-(C₁-C₄)-alkylaminosulfonyl-, or from (C₁-C₈)-alkylsulfonylamino-, (C₆-C₁₀)-arylsulfonylamino-, (C₆-C₁₀)-aryl-(C₁-C₄)-alkylsulfonylamino-, Het-sulfonylamino- and Het-(C₁-C₄)-alkylsulfonylamino-, and in each of these cases is substituted with zero, one or two identical or different substituents R¹⁰ which are selected from halogen (for example fluorine, chlorine and bromine, e.g. bromine), (C₁-C₈)-alkyl, (C₁-C₈)-alkoxy, (C₆-C₁₀)-aryloxy- and (C₆-C₁₀)-aryl-(C₁-C₄)-alkoxy-, where each of the aryl groups and Het groups in a group R¹¹ is unsubstituted or substituted as defined above.

The present invention encompasses all compounds of formula I in which one or more of the groups are defined as in any of the embodiments described above or have one or more of the specific denotations listed in their respective definitions or in the general explanations on the respective groups, all combinations of such specific denotations being among objects of the present invention. Moreover, any and all stereoisomeric forms of compounds of formula I, mixtures of (comprising two or more) compounds of formula I in any ratio, and physiologically tolerable salts of compounds of formula I are within the scope of the present invention. For example, one group of compounds is constituted by compounds of formula I in which:

n is 0;
R¹ is hydrogen;
R³ is hydrogen;
R⁵ is methyl, ethyl, furyl, thienyl, pyridyl or phenyl, where a furyl, thienyl, pyridyl or phenyl group is unsubstituted or substituted with one or more identical or different substituents R¹⁰; and
m, A, X, Y, Z, R², R⁴, R⁶, R⁷ and R¹⁰ are as defined in their general definitions or in any of the embodiments described above;
and any and all stereoisomeric forms thereof;
and mixtures thereof in any ratio;
and physiologically tolerable salts thereof.

Another group of compounds is constituted by compounds of formula I in which:

n is 0;
m is 0 or 1;
R¹ is hydrogen;
R² is hydrogen;
R³ is hydrogen;
R⁵ is methyl, ethyl, furyl, thienyl, pyridyl or phenyl, where a furyl, thienyl, pyridyl or phenyl group is unsubstituted or substituted with one or more identical or different substituents R¹⁰;
R⁶ is hydrogen;
R⁷ is hydrogen;
A, X, Y, Z, R⁴ and R¹¹ are as defined in their general definitions or in any of the embodiments described above;
and any and all stereoisomeric forms thereof;
and mixtures thereof in any ratio;
and physiologically tolerable salts thereof.

A further group of compounds of the invention is constituted by compounds of the formula Ia

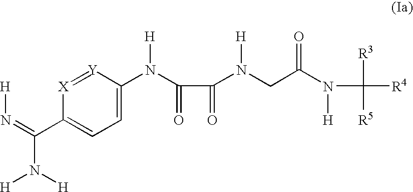

wherein X, Y, R³, R⁴ and R⁵ are as defined in their general definitions or in any of the embodiments described above;
and any and all stereoisomeric forms thereof;
and mixtures thereof in any ratio;
and physiologically tolerable salts thereof.

The compounds of formula I are reversible inhibitors of Factor VII polypeptides (in their activated form). Preferably, they are specific inhibitors of Factor VII polypeptides. As used herein, the term specific when used in reference to the inhibition of Factor VIIa activity means that a compound of the formula I can inhibit Factor VII activity without substantially inhibiting the activity of other specified proteases involved in the blood coagulation and/or the fibrinolysis pathway including, for example, Factor Xa, plasmin, thrombin, Factor IXa, Factor XIa, Factor XIIa and tissue-plasminogen activator (tPA) (using the same concentration of the inhibitor).

The present specification describes assays and methods (see below) for determining the inhibition constant (Kᵢ) for a Factor VII polypeptide as well as other specified proteases involved in the blood coagulation and/or the fibrinolysis pathway.

In preferred embodiments of the invention, the compounds of formula I exhibit at least one of the following:
a Kᵢ (activated Factor VII polypeptide) of 1/10 or less (such as 1/20 or less, 1/50 or less, 1/100 or less, 1/200 or less, 1/500 or less, from ½- 1/10, from 1/10- 1/20, from 1/10- 1/50, from 1/20- 1/100, from 1/50- 1/200, from 1/100- 1/500, from 1/100- 1/1000, from 1/200- 1/1000) of Kᵢ (Factor Xa) (using the same concentration of the inhibitor);
a Kᵢ (activated Factor VII polypeptide) of 1/10 or less (such as 1/20 or less, 1/50 or less, 1/100 or less, 1/200 or less, 1/500 or less, from ½- 1/10, from 1/10- 1/20, from 1/10- 1/50, from 1/20- 1/100, from 1/50- 1/200, from 1/100- 1/500, from 1/100- 1/1000, from 1/200- 1/1000) of Kᵢ (plasmin) (using the same concentration of the inhibitor);
a Kᵢ (activated Factor VII polypeptide) of 1/10 or less (such as 1/20 or less, 1/50 or less, 1/100 or less, 1/200 or less, 1/500 or less, from ½- 1/10, from 1/10- 1/20, from 1/10- 1/50, from 1/20- 1/100, from 1/50- 1/200, from 1/100- 1/500, from 1/100- 1/1000, from 1/200- 1/1000) of Kᵢ (thrombin) (using the same concentration of the inhibitor);
a Kᵢ (activated Factor VII polypeptide) of 1/10 or less (such as 1/20 or less, 1/50 or less, 1/100 or less, 1/200 or less, 1/500 or less, from ½- 1/10, from 1/10- 1/20, from 1/10- 1/50, from 1/20- 1/100, from 1/50- 1/200, from 1/100- 1/500, from 1/100- 1/1000, from 1/200- 1/1000) of Kᵢ (Factor IXa) (using the same concentration of the inhibitor);
a Kᵢ (activated Factor VII polypeptide) of 1/10 or less (such as 1/20 or less, 1/50 or less, 1/100 or less, 1/200 or less, 1/500 or less, from ½- 1/10, from 1/10- 1/20, from 1/10- 1/50, from 1/20- 1/100, from 1/50- 1/200, from 1/100- 1/500, from 1/100- 1/1000, from 1/200- 1/1000) of Kᵢ (Factor XIa) (using the same concentration of the inhibitor);
a Kᵢ (activated Factor VII polypeptide) of 1/10 or less (such as 1/20 or less, 1/50 or less, 1/100 or less, 1/200 or less, 1/500 or less, from ½- 1/10, from 1/10- 1/20, from 1/10- 1/50, from 1/20- 1/100, from 1/50- 1/200, from 1/100- 1/500, from 1/100- 1/1000, from 1/200- 1/1000) of Kᵢ (Factor XIIa) (using the same concentration of the inhibitor);
a Kᵢ (activated Factor VII polypeptide) of 1/10 or less (such as 1/20 or less, 1/50 or less, 1/100 or less, 1/200 or less, 1/500 or less, from ½- 1/10, from 1/10- 1/20, from 1/10- 1/50, from 1/20- 1/100, from 1/50- 1/200, from 1/100- 1/500, from 1/100- 1/1000, from 1/200- 1/1000) of Kᵢ (tPA) (using the same concentration of the inhibitor).

### Factor VII Polypeptides

As used herein, the term “Factor VII polypeptide” encompasses wild-type Factor VII (i.e. a polypeptide having the amino acid sequence disclosed in U.S. Pat. No. 4,784,950), as well as variants, derivatives and conjugates of Factor VII exhibiting substantially the same or improved biological activity relative to wild-type Factor VII. Moreover, the term is intended to encompass such polypeptides in their uncleaved (zymogen) form, as well as those that have been proteolytically processed to yield their respective bioactive forms (e.g. Factor VIIa). Typically, Factor VII is cleaved between amino acid residues 152 and 153 to yield Factor VIIa. Such variants, derivatives and conjugates of Factor VII may exhibit different properties relative to human Factor VII, including stability, phospholipid binding, altered specific activity, and the like.

“Factor VII” or “Factor VIIa” within the above definition also includes natural allelic variations that may exist and occur from one individual to another. Also, degree and location of glycosylation or other post-translation modifications may vary depending on the chosen host cells and the nature of the host cellular environment.

Variants of Factor VII in the present context include polypeptides having an amino acid sequence that differs from the sequence of wild-type Factor VII by insertion, deletion, or substitution of one or more amino acids (sequence variants).

The term “Factor VII derivative” as used herein, is intended to designate a FVII polypeptide exhibiting substantially the same or improved biological activity relative to wild-type Factor VII, in which one or more of the amino acids of the parent peptide have been genetically and/or chemically and/or enzymatically modified, e.g. by alkylation, glycosylation, PEGylation, acylation, ester formation or amide formation or the like. This includes but is not limited to PEGylated human Factor VIIa, cysteine-PEGylated human Factor VIIa and variants thereof. Non-limiting examples of Factor VII derivatives includes GlycoPegylated FVII derivatives as disclosed in WO 03/31464 and US patent applications US 20040043446, US 20040063911, US 20040142856, US 20040137557, and US 20040132640 (Neose Technologies, Inc.); FVII conjugates as disclosed in WO 01/04287, US patent application 20030165996, WO 01/58935, WO 03/93465 (Maxygen ApS) and WO 02/02764, US patent application 20030211094 (University of Minnesota). The term “PEGylated human Factor VIIa” means human Factor VIIa, having a PEG molecule conjugated to a human Factor VIIa polypeptide. It is to be understood, that the PEG molecule may be attached to any part of the Factor VIIa polypeptide including any amino acid residue or carbohydrate moiety of the Factor VIIa polypeptide. The term “cysteine-PEGylated human Factor VIIa” means Factor VIIa having a PEG molecule conjugated to a sulfhydryl group of a cysteine introduced in human Factor VIIa. Examples of Factor VII derivatives in the present context include, but are not limited to, derivatives of Factor VII polypeptides (such as Factor VII or Factor VIIa) wherein at least one organic substituent is bound to one or more amino acid residues, e.g. a derivative in which the Factor VII polypeptide is conjugated to a molecule such as a fatty acid (the derivative in question thus being an acylated Factor VII polypeptide derivative), an albumin, e.g. human serum albumin, or a water-soluble polymer such as a polyethyleneglycol (PEG), in order to achieve, e.g., protracted duration of Factor VII polypeptide activity after administration of the derivative in question to a subject (e.g. a human patient) in need thereof.

The term “improved biological activity” refers to FVII polypeptides with i) substantially the same or increased proteolytic activity compared to recombinant wild type human Factor VIIa or ii) to FVII polypeptides with substantially the same or increased TF binding activity compared to recombinant wild type human Factor VIIa or iii) to FVII polypeptides with substantially the same or increased half life in blood plasma compared to recombinant wild type human Factor VIIa.

Non-limiting examples of Factor VII (FVII) variants having, in the activated (FVIIa) form, substantially the same or increased proteolytic activity compared to recombinant wild type human Factor VIIa include S52A-FVIIa, S60A-FVIIa (Lino et al., Arch. Biochem. Biophys. 352: 182-192, 1998); FVIIa variants exhibiting increased proteolytic stability as disclosed in U.S. Pat. No. 5,580,560; Factor VIIa that has been proteolytically cleaved between residues 290 and 291 or between residues 315 and 316 (Mollerup et al., Biotechnol. Bioeng. 48:501-505, 1995); oxidized forms of Factor VIIa (Kornfelt et al., Arch. Biochem. Biophys. 363:43-54, 1999); FVII variants as disclosed in PCT/DK02/00189 (corresponding to WO 02/077218); and FVII variants exhibiting increased proteolytic stability as disclosed in WO 02/38162 (Scripps Research Institute); FVII variants having a modified Gla-domain and exhibiting an enhanced membrane binding as disclosed in WO 99/20767, U.S. Pat. No. 6,017,882 and U.S. Pat. No. 6,747,003, US patent application 20030100506 (University of Minnesota) and WO 00/66753, US patent applications US 20010018414, US 2004220106, and US 200131005, US patents U.S. Pat. No. 6,762,286 and U.S. Pat. No. 6,693,075 (University of Minnesota); and FVII variants as disclosed in WO 01/58935, U.S. Pat. No. 6,806,063, US patent application 20030096338 (Maxygen ApS), WO 03/93465 (Maxygen ApS), WO 04/029091 (Maxygen ApS), WO 04/083361 (Maxygen ApS), and WO 04/111242 (Maxygen ApS), as well as in WO 04/108763 (Canadian Blood Services).

Non-limiting examples of FVII variants having, in the activated (FVIIa) form, increased biological activity compared to wild-type FVIIa include FVII variants as disclosed in WO 01/83725, WO 02/22776, WO 02/077218, PCT/DK02/00635 (corresponding to WO 03/027147), Danish patent application PA 2002 01423 (corresponding to WO 04/029090), Danish patent application PA 2001 01627 (corresponding to WO 03/027147); WO 02/38162 (Scripps Research Institute); and FVIIa variants with enhanced activity as disclosed in 3P 2001061479 (Chemo-Sero-Therapeutic Res Inst.).

Examples of variants of factor VII include, without limitation, P10Q-FVII, K32E-FVII, P10Q/K32E-FVII, L305V-FVII, L305V/M306D/D309S-FVII, L305I-FVII, L305T-FVII, F374P-FVII, V158T/M298Q-FVII, V158D/E296V/M298Q-FVII, K337A-FVII, M298Q-FVII, V158D/M298Q-FVII, L305V/K337A-FVII, V158D/E296V/M298Q/L305V-FVII, V158D/E296V/M298Q/K337A-FVII, V158D/E296V/M298Q/L305V/K337A-FVII, K157A-FVII, E296V-FVII, E296V/M298Q-FVII, V158D/E296V-FVII, V158D/M298K-FVII, and S336G-FVII, L305V/K337A-FVII, L305V/V158D-FVII, L305V/E296V-FVII, L305V/M298Q-FVII, L305V/V158T-FVII, L305V/K337A/V158T-FVII, L305V/K337A/M298Q-FVII, L305V/K337A/E296V-FVII, L305V/K337A/V158D-FVII, L305V/V158D/M298Q-FVII, L305V/V158D/E296V-FVII, L305V/V158T/M298Q-FVII, L305V/V158T/E296V-FVII, L305V/E296V/M298Q-FVII, L305V/V158D/E296V/M298Q-FVII, L305V/V158T/E296V/M298Q-FVII, L305V/V158T/K337A/M298Q-FVII, L305V/V158T/E296V/K337A-FVII, L305V/V158D/K337A/M298Q-FVII, L305V/V158D/E296V/K337A-FVII, L305V/V158D/E296V/M298Q/K337A-FVII, L305V/V158T/E296V/M298Q/K337A-FVII, S314E/K316H-FVII, S314E/K316Q-FVII, S314E/L305V-FVII, S314E/K337A-FVII, S314E/V158D-FVII, S314E/E296V-FVII, S314E/M298Q-FVII, S314E/V158T-FVII, K316H/L305V-FVII, K316H/K337A-FVII, K316H/V158D-FVII, K316H/E296V-FVII, K316H/M298Q-FVII, K316H/V158T-FVII, K316Q/L305V-FVII, K316Q/K337A-FVII, K316Q/V158D-FVII, K316Q/E296V-FVII, K316Q/M298Q-FVII, K316Q/V158T-FVII, S314E/L305V/K337A-FVII, S314E/L305V/V158D-FVII, S314E/L305V/E296V-FVII, S314E/L305V/M298Q-FVII, S314E/L305V/V158T-FVII, S314E/L305V/K337A/V158T-FVII, S314E/L305V/K337A/M298Q-FVII, S314E/L305V/K337A/E296V-FVII, S314E/L305V/K337A/V158D-FVII, S314E/L305V/V158D/M298Q-FVII, S314E/L305V/V158D/E296V-FVII, S314E/L305V/V158T/M298Q-FVII, S314E/L305V/V158T/E296V-FVII, S314E/L305V/E296V/M298Q-FVII, S314E/L305V/V158D/E296V/M298Q-FVII, S314E/L305V/V158T/E296V/M298Q-FVII, S314E/L305V/V158T/K337A/M298Q-FVII, S314E/L305V/V158T/E296V/K337A-FVII, S314E/L305V/V158D/K337A/M298Q-FVII, S314E/L305V/V158D/E296V/K337A-FVII, S314E/L305V/V158D/E296V/M298Q/K337A-FVII, S314E/L305V/V158T/E296V/M298Q/K337A-FVII, K316H/L305V/K337A-FVII, K316H/L305V/V158D-FVII, K316H/L305V/E296V-FVII, K316H/L305V/M298Q-FVII, K316H/L305V/V158T-FVII, K316H/L305V/K337A/V158T-FVII, K316H/L305V/K337A/M298Q-FVII, K316H/L305V/K337A/E296V-FVII, K316H/L305V/K337A/V158D-FVII, K316H/L305V/V158D/M298Q-FVII, K316H/L305V/V158D/E296V-FVII, K316H/L305V/V158T/M298Q-FVII, K316H/L305V/V158T/E296V-FVII, K316H/L305V/E296V/M298Q-FVII, K316H/L305V/V158D/E296V/M298Q-FVII, K316H/L305V/V158T/E296V/M298Q-FVII, K316H/L305V/V158T/K337A/M298Q-FVII, K316H/L305V/V158T/E296V/K337A-FVII, K316H/L305V/V158D/K337A/M298Q-FVII, K316H/L305V/V158D/E296V/K337A-FVII, K316H/L305V/V158D/E296V/M298Q/K337A-FVII, K316H/L305V/V158T/E296V/M298Q/K337A-FVII, K316Q/L305V/K337A-FVII, K316Q/L305V/V158D-FVII, K316Q/L305V/E296V-FVII, K316Q/L305V/M298Q-FVII, K316Q/L305V/V158T-FVII, K316Q/L305V/K337A/V158T-FVII, K316Q/L305V/K337A/M298Q-FVII, K316Q/L305V/K337A/E296V-FVII, K316Q/L305V/K337A/V158D-FVII, K316Q/L305V/V158D/M298Q-FVII, K316Q/L305V/V158D/E296V-FVII, K316Q/L305V/V158T/M298Q-FVII, K316Q/L305V/V158T/E296V-FVII, K316Q/L305V/E296V/M298Q-FVII, K316Q/L305V/V158D/E296V/M298Q-FVII, K316Q/L305V/V158T/E296V/M298Q-FVII, K316Q/L305V/V158T/K337A/M298Q-FVII, K316Q/L305V/V158T/E296V/K337A-FVII, K316Q/L305V/V158D/K337A/M298Q-FVII, K316Q/L305V/V158D/E296V/K337A-FVII, K316Q/L305V/V158D/E296V/M298Q/K337A-FVII, K316Q/L305V/V158T/E296V/M298Q/K337A-FVII, F374Y/K337A-FVII, F374Y/V158D-FVII, F374Y/E296V-FVII, F374Y/M298Q-FVII, F374Y/V158T-FVII, F374Y/S314E-FVII, F374Y/L305V-FVII, F374Y/L305V/K337A-FVII, F374Y/L305V/V158D-FVII, F374Y/L305V/E296V-FVII, F374Y/L305V/M298Q-FVII, F374Y/L305V/V158T-FVII, F374Y/L305V/S314E-FVII, F374Y/K337A/S314E-FVII, F374Y/K337A/V158T-FVII, F374Y/K337A/M298Q-FVII, F374Y/K337A/E296V-FVII, F374Y/K337A/V158D-FVII, F374Y/V158D/S314E-FVII, F374Y/V158D/M298Q-FVII, F374Y/V158D/E296V-FVII, F374Y/V158T/S314E-FVII, F374Y/V158T/M298Q-FVII, F374Y/V158T/E296V-FVII, F374Y/E296V/S314E-FVII, F374Y/S314E/M298Q-FVII, F374Y/E296V/M298Q-FVII, F374Y/L305V/K337A/V158D-FVII, F374Y/L305V/K337A/E296V-FVII, F374Y/L305V/K337A/M298Q-FVII, F374Y/L305V/K337A/V158T-FVII, F374Y/L305V/K337A/S314E-FVII, F374Y/L305V/V158D/E296V-FVII, F374Y/L305V/V158D/M298Q-FVII, F374Y/L305V/V158D/S314E-FVII, F374Y/L305V/E296V/M298Q-FVII, F374Y/L305V/E296V/V158T-FVII, F374Y/L305V/E296V/S314E-FVII, F374Y/L305V/M298Q/V158T-FVII, F374Y/L305V/M298Q/S314E-FVII, F374Y/L305V/V158T/S314E-FVII, F374Y/K337A/S314E/V158T-FVII, F374Y/K337A/S314E/M298Q-FVII, F374Y/K337A/S314E/E296V-FVII, F374Y/K337A/S314E/V158D-FVII, F374Y/K337A/V158T/M298Q-FVII, F374Y/K337A/V158T/E296V-FVII, F374Y/K337A/M298Q/E296V-FVII, F374Y/K337A/M298Q/V158D-FVII, F374Y/K337A/E296V/V158D-FVII, F374Y/V158D/S314E/M298Q-FVII, F374Y/V158D/S314E/E296V-FVII, F374Y/V158D/M298Q/E296V-FVII, F374Y/V158T/S314E/E296V-FVII, F374Y/V158T/S314E/M298Q-FVII, F374Y/V158T/M298Q/E296V-FVII, F374Y/E296V/S314E/M298Q-FVII, F374Y/L305V/M298Q/K337A/S314E-FVII, F374Y/L305V/E296V/K337A/S314E-FVII, F374Y/E296V/M298Q/K337A/S314E-FVII, F374Y/L305V/E296V/M298Q/K337A-FVII, F374Y/L305V/E296V/M298Q/S314E-FVII, F374Y/V158D/E296V/M298Q/K337A-FVII, F374Y/V158D/E296V/M298Q/S314E-FVII, F374Y/L305V/V158D/K337A/S314E-FVII, F374Y/V158D/M298Q/K337A/S314E-FVII, F374Y/V158D/E296V/K337A/S314E-FVII, F374Y/L305V/V158D/E296V/M298Q-FVII, F374Y/L305V/V158D/M298Q/K337A-FVII, F374Y/L305V/V158D/E296V/K337A-FVII, F374Y/L305V/V158D/M298Q/S314E-FVII, F374Y/L305V/V158D/E296V/S314E-FVII, F374Y/V158T/E296V/M298Q/K337A-FVII, F374Y/V158T/E296V/M298Q/S314E-FVII, F374Y/L305V/V158T/K337A/S314E-FVII, F374Y/V158T/M298Q/K337A/S314E-FVII, F374Y/V158T/E296V/K337A/S314E-FVII, F374Y/L305V/V158T/E296V/M298Q-FVII, F374Y/L305V/V158T/M298Q/K337A-FVII, F374Y/L305V/V158T/E296V/K337A-FVII, F374Y/L305V/V158T/M298Q/S314E-FVII, F374Y/L305V/V158T/E296V/S314E-FVII, F374Y/E296V/M298Q/K337A/V158T/S314E-FVII, F374Y/V158D/E296V/M298Q/K337A/S314E-FVII, F374Y/L305V/V158D/E296V/M298Q/S314E-FVII, F374Y/L305V/E296V/M298Q/V158T/S314E-FVII, F374Y/L305V/E296V/M298Q/K337A/V158T-FVII, F374Y/L305V/E296V/K337A/V158T/S314E-FVII, F374Y/L305V/M298Q/K337A/V158T/S314E-FVII, F374Y/L305V/V158D/E296V/M298Q/K337A-FVII, F374Y/L305V/V158D/E296V/K337A/S314E-FVII, F374Y/L305V/V158D/M298Q/K337A/S314E-FVII, F374Y/L305V/E296V/M298Q/K337A/V158T/S314E-FVII, F374Y/L305V/V158D/E296V/M298Q/K337A/S314E-FVII, S52A-Factor VII, S60A-Factor VII; R152E-Factor VII, S344A-Factor VII, T106N-FVII, K143N/N145T-FVII, V253N-FVII, R290N/A292T-FVII, G291N-FVII, R315N/V317T-FVII, K143N/N145T/R315N/V317T-FVII; and FVII having substitutions, additions or deletions in the amino acid sequence from 233Thr to 240Asn; FVII having substitutions, additions or deletions in the amino acid sequence from 304Arg to 329Cys; and FVII having substitutions, additions or deletions in the amino acid sequence from 153Ile to 223Arg.

Thus, substitution variants in a factor VII polypeptide include, without limitation substitutions in positions P10, K32, L305, M306, D309, L305, L305, F374, V158, M298, V158, E296, K337, M298, M298, S336, S314, K316, K316, F374, S52, S60, R152, S344, T106, K143, N145, V253, R290, A292, G291, R315, V317, and substitutions, additions or deletions in the amino acid sequence from T233 to N240 or from R304 to C329; or from 1153 to R223, or combinations thereof, in particular variants such as P10Q, K32E, L305V, M306D, D309S, L305I, L305T, F374P, V158T, M298Q, V158D, E296V, K337A, M298Q, M298K, S336G, S314E, K316H, K316Q, F374Y, S52A, S60A, R152E, S344A, T106N, K143N, N145T, V253N, R290N, A292T, G291N, R315N, V317T, and substitutions, additions or deletions in the amino acid sequence from T233 to N240, or from R304 to C329, or from 1153 to R223, or combinations thereof.

The biological activity of Factor VII (as Factor VIIa) in blood clotting derives from its ability to (i) bind to tissue factor (TF) and (ii) catalyze the proteolytic cleavage of Factor IX or Factor X to produce activated Factor IX or X (Factor IXa or Xa, respectively). Human Factor VIIa biological activity may be quantified by an assay measuring the ability of a preparation to promote blood clotting using Factor VII-deficient plasma and thromboplastin, as described, e.g., in U.S. Pat. No. 5,997,864. In this assay, biological activity is expressed as the reduction in clotting time relative to a control sample and is converted to “Factor VII units” by comparison with a pooled human serum standard containing 1 unit/ml Factor VII activity. Alternatively, Factor VII polypeptides may also be assayed for specific activities (“clot activity”) by using a one-stage coagulation assay. For this purpose, the sample to be tested is diluted in 50 mM PIPES-buffer (pH 7.5), 0.1% BSA and 40 μl is incubated with 40 μl of Factor VII deficient plasma and 80 μl of human recombinant tissue factor containing 10 mM Ca²⁺ and synthetic phospholipids. Coagulation times (clotting times) are measured and compared to a standard curve using a reference standard in a parallel line assay. Alternatively, Factor VIIa biological activity may be quantified by (i) measuring the ability of Factor VIIa to produce Factor Xa in a system comprising TF embedded in a lipid membrane and Factor X. (Persson et al., J. Biol. Chem. 272:19919-19924, 1997); (ii) measuring Factor X hydrolysis in an aqueous system; (iii) measuring its physical binding to TF using an instrument based on surface plasmon resonance (Persson, FEBS Letts. 413:359-363, 1997) and (iv) measuring hydrolysis of a synthetic substrate.

Factor VII variants having, in the activated (FVIIa) form, substantially the same or improved biological activity relative to wild-type Factor VIIa encompass those that exhibit at least about 10%, preferably at least about 25%, more preferably at least about 50%, even more preferably at least about 75% and most preferably at least about 90% of the specific activity of Factor VIIa that has been produced in the same cell type, when tested in one or more of a clotting assay, proteolysis assay or TF binding assay as described above.

### Preparation of Compounds of the Invention

Compounds of formula I according to the invention may be synthesized using procedures analogous to those described in U.S. Pat. No. 6,743,790 B2, and analogous to those described in Example 1 herein, and a person of ordinary skill in the art will in general be able to modify the procedures described therein, without undue experimentation, in order to prepare compounds of the present invention using readily available starting materials.

### Pharmaceutical Formulation and Administration of Factor VII Polypeptides

In general, an aqueous, liquid Factor VII polypeptide formulation or composition of the invention (aqueous pharmaceutical composition of the invention) will—irrespective of whether the aqueous formulation is present in aqueous liquid form from the start, or is produced by dissolution/reconstitution of a substantially solid formulation (e.g. a lyophilized preparation) by addition of water or another aqueous carrier or vehicle—in general, suitably be administered parenterally, i.e., intravenously, subcutaneously, or intramuscularly, or by continuous or pulsatile infusion.

For use in human subjects, Factor VII polypeptide compositions of the invention for parenteral administration will, in addition to a compound of formula I or a physiologically tolerable salt thereof in an appropriate concentration, normally comprise the Factor VII polypeptide in combination with, preferably dissolved in, a pharmaceutically acceptable aqueous carrier. A variety of aqueous carriers may be used, such as water, buffered water, 0.4% saline, 0.3% glycine and the like. Factor VII polypeptides in the context of the invention may also be formulated into liposome preparations for delivery or targeting to the sites of injury. Liposome preparations are generally described in, e.g., U.S. Pat. No. 4,837,028, U.S. Pat. No. 4,501,728 and U.S. Pat. No. 4,975,282. The compositions may be sterilised by conventional, well-known sterilisation techniques. The resulting aqueous solutions may be packaged for use as such, or they may be filtered under aseptic conditions and lyophilized, the lyophilized preparation being combined with sterile water or a sterile aqueous solution (carrier, vehicle) prior to administration. The compositions may further contain pharmaceutically acceptable auxiliary substances as required to approximate physiological conditions and/or to enhance the chemical and/or physical stability of the composition. These include:

pH-adjusting and/or buffering agents, e.g. citrate (sodium or potassium), acetate (ammonium, sodium or calcium), histidine (L-histidine), malate, phosphate (sodium or potassium), tartaric acid, succinic acid, MES (2-N-morpholino-ethanesulfonic acid), HEPES (4-(2-hydroxy-ethyl)-piperazine-1-ethane-sulfonic acid), imidazole, TRIS [tris(hydroxymethyl)aminomethane], lactate, and glutamate. The buffer concentration range is chosen to maintain the preferred pH of the solution. The buffering agent may also be a mixture of two or more buffering agents, e.g. a mixture of two such agents, such that the mixture is able to provide a pH value in the specified range. In one embodiment, the buffer is a mixture of citrate and at least one of the buffers acetate (ammonium, sodium or calcium), histidine (L-histidine), malate, phosphate (sodium or potassium), tartaric acid, succinic acid, MES, HEPES, imidazole, TRIS, lactate and glutamate. The total concentration of buffer agent(s) is typically in the range of from about 1 mM to about 100 mM, such as from about 1 mM to about 50 mM, often from about 1 mM to about 25 mM, e.g. from about 2 mM to about 20 mM.

calcium salts: the compositions—whether initially in liquid, freeze-dried or reconstituted form—may optionally contain a calcium salt. The calcium salt may be present in a low concentration, such as, e.g., from about 0.1 mM to about 5 mM; it may be present in a medium concentration, such as, e.g., from about 5 mM to about 15 mM; or it may be present in a higher concentration, such as, e.g., from about 15 mM to about 1000 mM. In one aspect, the calcium salt is selected from: calcium chloride, calcium acetate, calcium gluconate and calcium laevulate, and mixtures of two or more thereof. Alternatively, the concentration of calcium ions in the composition may be below 0.1 mM.

tonicity-adjusting agents (tonicity-modifying substances which contribute to the osmolality of the formulation), e.g. amino acids, small peptides (having, e.g., from 2 to 5 amino acid residues), neutral salts, mono- or disaccharides, polysaccharides, sugar alcohols, or mixtures of at least two of such substances. Specific examples include, but are not limited to, sodium chloride, potassium chloride, sodium citrate, sucrose, glucose and mannitol. The concentration of tonicity-adjusting agent is adjusted to near isotonicity, depending on the other ingredients present in the formulation. In general, tonicity-adjusting agents are incorporated in a concentration of from about 1 to about 500 mM, such as from about 1 to about 300 mM, often from about 10 to about 200 mM, e.g. from about 20 to about 150 mM, depending on the other ingredients present. Neutral salts such as, e.g., sodium chloride or potassium chloride may be used. The term “neutral salt” indicates a salt that is substantially neither acidic nor basic, i.e. has little or no effect on formulation pH when dissolved;

surfactants, typically a non-ionic surfactant, suitably of the polysorbate or Tween™ type (e.g. Polysorbate-20 or 80, or Tween™ 80), or of the poloxamer or Pluronic™ type (e.g. Poloxamer™ 188 or 407). The amount of surfactant incorporated may typically range from about 0.005 to about 1% weight/weight (w/w), with amounts of from about 0.005 to about 0.1% w/w, such as from about 0.005 to 0.02% w/w, typically being preferred. In some situations, relatively high concentrations, e.g. up to about 0.5% w/w, are desirable to maintain protein stability. However, the levels of surfactant used in actual practice are customarily limited by clinical practice;

antioxidants, e.g. ascorbic acid, cysteine, homocysteine, cystine, cysstathionine, methionine, glutathione, or peptides containing cysteine or methionine; methionine, in particular L-methionine, is typically a very suitable antioxidant. An antioxidant is typically incorporated in a concentration of from about 0.1 to about 2 mg/ml;

preservatives (included in the formulation to retard microbial growth, thereby permitting, for example, “multiple use” packaging of the FVII polypeptide), e.g. phenol, benzyl alcohol, ortho-cresol, meta-cresol, para-cresol, methylparaben, propylparaben, benzalconium chloride or benzethonium chloride. A preservative is typically incorporated in a concentration of from about 0.1 to about 2 mg/ml;

The concentration of Factor VII polypeptide in the compositions can vary widely, typically from about 0.01% w/w to about 2% w/w (i.e. from about 0.1 mg/ml to about 20 mg/ml), such as from about 0.05% w/w to about 1.5% w/w (i.e. from about 0.5 mg/l to about 15 mg/ml), e.g. from about 0.05% w/w to about 1% w/w (i.e. from about 0.5 mg/ml to about 10 mg/ml), and will be selected primarily on the basis of fluid volumes, viscosities, etc., in accordance with the particular mode of administration selected. In the case of Factor VIIa, concentration is frequently expressed as mg/ml or as International units/ml (IU/ml). 1 mg of FVIIa usually corresponds to 43000-56000 IU or more.

The concentration of a compound (or compounds) of formula I of the invention in a liquid, aqueous pharmaceutical composition of the invention will typically be at least 1 μM. The desirable (or necessary) concentration typically depends on the selected compound (or compounds), more specifically on the binding affinity of the selected compound(s) to the Factor VII polypeptide. In various embodiments, the compound of formula I may be present in a concentration of at least 5 μM, at least 10 μM, at least 20 μM, at least 50 μM, at least 100 μM, at least 150 μM, at least 250 μM, at least 500 μM, at least 1 mM, at least 2 mM, at least 4 mM, at least 5 mM, at least 8 mM, at least 9 mM, at least 10 mM, at least 15 mM, or at least 20 mM, such as, e.g., in the range of 1-10000 μM, 10-10000 μM, 20-10000 μM, 50-10000 μM, 10-5000 μM, 10-2000 μM, 20-5000 μM, 20-2000 μM, 50-5000 μM, 0.1-100 mM, 0.1-75 mM, 0.1-50 mM, 0.1-10 mM, 0.2-75 mM, 0.2-50 mM, 0.2-20 mM, 0.5-75 mM or 0.5-50 mM.

In various embodiments, the molar ratio between the compound of formula I and FVII polypeptide may be: above 0.1, above 0.5, above 1, above 2, above 5, above 10, above 25, above 100, above 250, above 1000, above 2500, or above 5000, such as, e.g., in the range of 0.1-10000, 0.1-5000, 0.1-2500, 0.1-1000, 0.1-250, 0.1-100, 0.1-25, 0.1-10, 0.5-10000, 0.5-5000, 0.5-2500, 0.5-1000, 0.5-250, 0.5-100, 0.5-25, 0.5-10, 1-10000, 1-5000, 1-2500, 1-1000, 1-250, 1-100; 1-25; 1-10, 10-10000, 10-5000, 10-250, 1000-10000, or 1000-5000.

Methods for preparing parenterally administrable compositions will be known or apparent to those skilled in the art, and are described in more detail in, for example, Remington's Pharmaceutical Sciences, 18^{th} edition, Mack Publishing Company, Easton, Pa. (1990).

For treatment in connection with deliberate interventions (e.g. surgical procedures), Factor VII polypeptides will typically be administered within about 24 hours prior to performing the intervention, and for as much as 7 days or more thereafter. Administration as a coagulant can be by a variety of routes as described herein. The dose of Factor VII polypeptide (e.g. rhFVIIa) will normally range from about 0.05 mg/day to 500 mg/day, preferably from about 1 mg/day to about 200 mg/day, and more preferably from about 10 mg/day to about 175 mg/day for a 70 kg subject as loading and maintenance doses, depending on the weight of the subject and the severity of the condition.

Compositions containing Factor VII polypeptides may be administered for prophylactic and/or therapeutic treatments. In therapeutic applications, compositions are administered to a subject already suffering from a condition, as described above, in an amount sufficient to cure, alleviate or partially arrest the condition and its complications. An amount adequate to accomplish this is defined as a “therapeutically effective amount”. As will be understood by the person skilled in the art, amounts effective for this purpose will depend on the severity of the condition or injury, as well as on the body weight and general physical condition of the subject.

It should be borne in mind that pharmaceutical compositions of FVII polypeptides (e.g. rhFVIIa) are generally employed in connection with life-threatening or potentially life-threatening medical conditions or states, and in such circumstances—in view of the general advantages associated with minimizing quantities of extraneous substances, and taking into account the general lack of immunogenicity of human Factor VII polypeptides—it is possible and may be felt desirable by the treating physician to administer a substantial excess of the Factor VII polypeptide in question.

In prophylactic applications, compositions containing a Factor VII polypeptide are administered to a subject susceptible to, or otherwise at risk of, a disease state or injury in order to enhance the subject's own coagulative capability. The dosage employed for such purposes (which may be termed a “prophylactically effective dose”) will once again depend on the subject's body weight and general state of health, but will once again generally range from about 0.05 mg/day to about 500 mg/day, more commonly from about 1.0 mg/day to about 200 mg/day for a 70-kilogram subject.

In the case, specifically, of administration of rhFVIIa to human subjects, dosage levels have generally been in the range of about 90-120 μg/kg body weight per dose. However, there is a current preference for somewhat higher doses, e.g. doses in excess of 150 μg/kg body weight, and in some cases doses of about 250-300 μg/kg.

Single or multiple administration of the composition in question may be carried out using dose levels and dosing regimens selected by the treating physician. For out-patients requiring daily maintenance levels, a Factor VII polypeptide may be administered by continuous infusion, e.g. using a portable pump system.

Local administration of a Factor VII polypeptide, e.g. topical application, may be carried out, e.g., by spraying, by perfusion, by use of a double balloon catheter or a stent, by incorporation into vascular grafts or stents, in the form of hydrogels to coat balloon catheters, or by other well established methods. In any event, the pharmaceutical composition in question should provide a quantity of Factor VII polypeptide which is adequate to effectively treat the subject.

### Further Aspects of the Invention

As already indicated to some extent above, further aspects of the present invention include the following:
a pharmaceutical composition (e.g. a liquid, aqueous pharmaceutical composition) comprising: one or more compounds, or physiologically tolerable salts thereof, according to the invention; and a Factor VII polypeptide (e.g. wild-type human FVIIa, such as rhFVIIa);
a method of preparing a composition comprising a Factor VII polypeptide (e.g. wild-type human FVIIa, such as rhFVIIa), comprising: adding a compound, or a physiologically tolerable salt thereof, according to the invention to a sample containing the Factor VII polypeptide; or adding the Factor VII polypeptide to a sample containing a compound, or a physiologically tolerable salt thereof, according to the invention; in a method of this type, the compound or salt thereof and/or the Factor VII polypeptide may be present in a liquid, aqueous medium.
a pharmaceutical composition prepared by a method according to the invention for preparing such a composition;
a method of inhibiting a Factor VII polypeptide (e.g. wild-type FVIIa, such as rhFVIIa), comprising: adding a compound, or a physiologically tolerable salt thereof, according to the invention to a sample containing the Factor VII polypeptide; or adding the Factor VII polypeptide to a sample containing a compound, or a physiologically tolerable salt thereof, according to the invention; in a method of this type, the compound or salt thereof and/or the Factor VII polypeptide may be present in a liquid, aqueous medium.
the use of a pharmaceutical composition according to the invention in the treatment of a condition or disorder against which the Factor VII polypeptide in question is effective

### EXAMPLE SECTION

### Abbreviations

Boc₂O Di(1,1-dimethylethyl)-dicarbonate
BSA Bovine serum albumin

### DIPEA N,N-Diisopropyl-N-ethylamine

### DMF N,N-Dimethylformamide

DMSO Dimethyl sulfoxide
EDCl 1-(3-Dimethylaminopropyl)-3-ethylcarbodiimide (as hydrochloride salt)

### HOBt N-Hydroxybenzotriazole

PEG Polyethylene glycol
PIPES 1,4-piperazinebis(ethanesulfonic acid)

### TEA Triethylamine

### THF Tetrahydrofuran

TFA Trifluoroacetic acid

### Example 1

### Synthesis of (S)-N-(5-carbamimidoyl-thiophen-2-yl)-N′-{[1-(3-methoxyphenyl)-ethylcarbamoyl]-methyl}-oxalamide

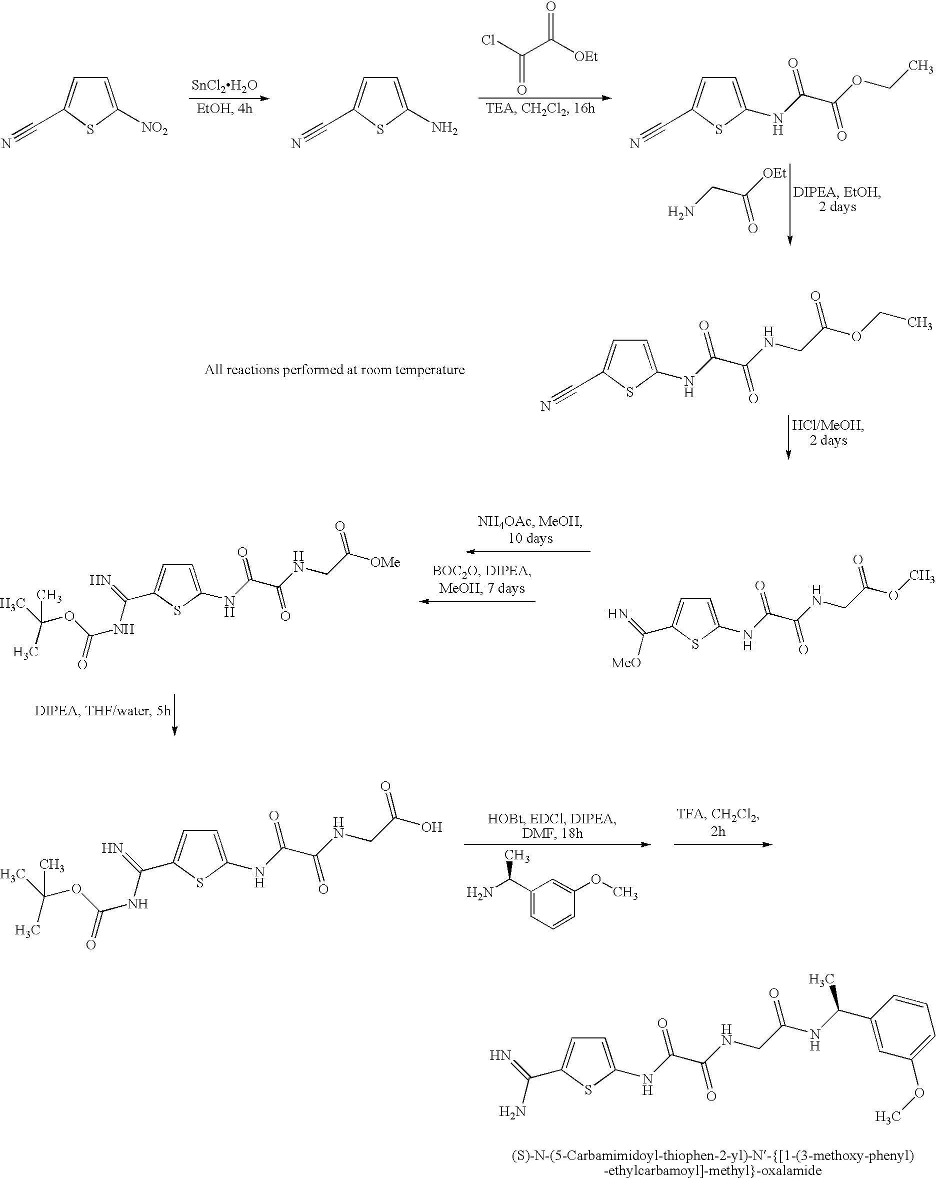

Additional examples of compounds of formula I (Examples 2-6) are listed in Table 1, below. All the compounds of Examples 2-6 may be synthesised by procedures analogous to that of Example 1, above, using readily available starting materials:

TABLE 1

| Example | Structure | Name |
|---|---|---|
| 2 | 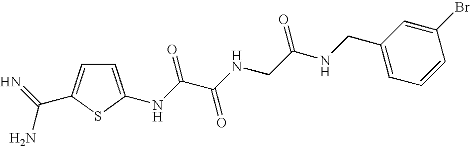 | N-[(3-Bromo-benzylcarbamoyl)-methyl]-N′-(5-carbamimidoyl-thiophen-2-yl)-oxalamide |
| 3 | 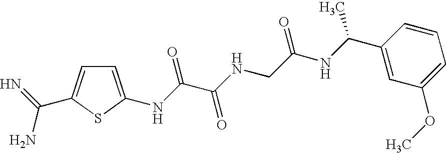 | (R)-N-(5-Carbamimidoyl-thiophen-2-yl)-N′-{[1-(3-methoxy-phenyl)-ethylcarbamoyl]-methyl}-oxalamide |
| 4 | 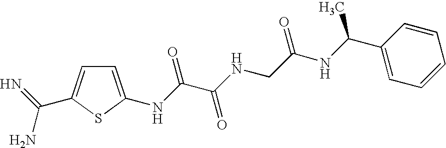 | (S)-N-(5-Carbamimidoyl-thiophen-2-yl)-N′-[(1-phenyl-ethylcarbamoyl)-methyl]-oxalamide |
| 5 | 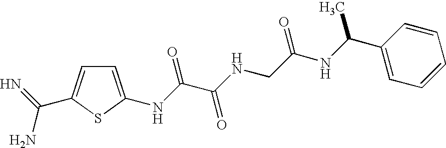 | (R)-N-(5-Carbamimidoyl-thiophen-2-yl)-N′-[(1-phenyl-ethylcarbamoyl)-methyl]-oxalamide |
| 6 | 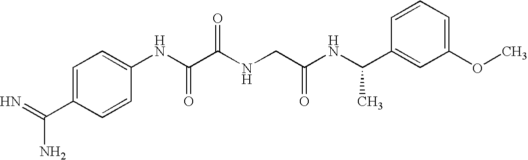 | (S)-N-(4-Carbamimidoyl-phenyl)-N′-{[1-(3-methoxy-phenyl)-ethylcarbamoyl]-methyl}-oxalamide |

### Assay Procedures

### (1) Enzyme Inhibition—General

The ability of compounds of formula I to inhibit Factor VIIa, or other enzymes/Factors such as Factor Xa, thrombin, plasmin or trypsin, is assessed by determining the concentration of the compound of formula I that inhibits the activity of the enzyme/Factor in question by 50%, i.e. the IC₅₀ value, which is related to the inhibition constant Ki. Purified enzymes are used in chromogenic assays. The concentration of inhibitor (compound of formula I) that causes a 50% decrease in the rate of hydrolysis of an appropriate substrate is determined by linear regression after plotting the relative rates of hydrolysis (compared to the uninhibited control) versus the log of the concentration of the compound of formula I.

For calculating the inhibition constant Ki, correction of the IC₅₀ value for competition with substrate is taken account of by using the formula:

Ki=IC₅₀/{1+(substrate concentration/Km)},

where Km is the Michaelis-Menten constant [Chen and Prusoff, Biochem. Pharmacol. 22 (1973), 3099-3108; I. H. Segal, Enzyme Kinetics, 1975, John Wiley & Sons, New York, 100-125; both of which references are incorporated herein in their entirety by reference].

### (1)a Factor VIIa (FVIIa) Assay

The inhibitory activity [expressed as inhibition constant Ki(FVIIa)] of compounds of formula I towards Factor VIIa/tissue factor activity may be determined using a chromogenic assay essentially as described previously [3. A. Ostrem et al., Biochemistry 37 (1998) 1053-1059, which reference is incorporated herein in its entirety by reference). Kinetic assays are conducted at 25° C. in half-area microtiter plates (Costar Corp., Cambridge, Mass.) using a kinetic plate reader (Molecular Devices Spectramax 250). In a typical assay, 25 μl rhFVIIa and TF (final concentrations 5 nM and 10 nM, respectively) are combined with 40 μl of inhibitor dilutions in 10% DMSO/TBS-PEG buffer (50 mM Tris, 15 mM NaCl, 5 mM CaCl₂, 0.05% PEG 8000, pH 8.15). Following a 15 minute preincubation period, the assay is initiated by the addition of 35 μl of the chromogenic substrate S-2288 (D-Ile-Pro-Arg-p-nitroanilide, Pharmacia Hepar Inc., 500 μM final concentration).

The following assays may be employed to investigate the possible inhibition of certain other coagulation enzymes and other serine proteases by compounds of formula I, and thus to determine the specificity of compounds of formula I.

### (I)b Factor Xa Assay

A TBS-PEG buffer of composition 50 mM Tris-Cl, pH 7.8, 200 mM NaCl, 0.05% (w/v) PEG-8000, 0.02% (w/v) NaN₃) is used for this assay. The IC₅₀ is determined by combining:

25 μl human factor Xa (Enzyme Research Laboratories, Inc.; South Bend, Ind.) in TBS-PEG buffer;
40 μl 10% (v/v) DMSO in TBS-PEG buffer (uninhibited control) or various concentrations of the compound to be tested diluted in 10% (v/v) DMSO in TBS-PEG; and
substrate S-2765 [N(α)-benzyloxycarbonyl-D-Arg-Gly-L-Arg-p-nitroanilide; Kabi Pharmacia, Inc.; Franklin, Ohio) in TBS-PEG.

in appropriate wells of a Costar half-area microtiter plate.

The assay is performed by pre-incubating the compound of formula I plus enzyme for 10 min. The assay is then initiated by adding substrate to obtain a final volume of 100 μl. The initial velocity of chromogenic substrate hydrolysis is measured by the change in absorbance at 405 nm using a Bio-tek Instruments kinetic plate reader (Ceres UV900HDi) at 25° C. during the linear portion of the time course (usually 1.5 min after addition of substrate). The enzyme concentration is 0.5 nM, and substrate concentration is 140 μM.

### (1)c Thrombin Assay

TBS-PEG buffer is likewise used in this assay. The IC₅₀ is determined as above for the Factor Xa assay, except that the substrate employed is S-2366 (L-PyroGlu-L-Pro-L-Arg-p-nitroanilide; Kabi) and the enzyme is human thrombin (Enzyme Research Laboratories, Inc.; South Bend, Ind.). The enzyme concentration is 175 μM.

### (1)d Plasmin Assay

TBS-PEG buffer is likewise used in this assay. The IC₅₀ is determined as described above for the Factor Xa assay, except that the substrate employed is S-2251 (D-Val-L-Leu-L-Lys-p-nitroanilide; Kabi) and the enzyme is human plasmin (Kabi). The enzyme concentration is 5 nM and the substrate concentration is 300 μM.

### (1)e Trypsin Assay

TBS-PEG buffer containing 10 mM CaCl₂ is used for this assay. The IC₅₀ is determined as described above for the Factor Xa assay, except that the substrate employed is BAPNA (benzoyl-L-Arg-p-nitroanilide; Sigma Chemical Co.; St. Louis, Mo.) and the enzyme is bovine pancreatic trypsin (Type XIII, TPCK treated; Sigma). The enzyme concentration is 50 nM and the substrate concentration is 300 μM.

### (2)a Amidolytic Assays for FIXa and tPA.

Enzymes and substrates are from American Diagnostica; FIXa (cat no 449b), FIXa sub-strate (cat no 299F), tPA (cat no 170) and tPA substrate (cat no 444LF).

Hydrolysis of substrates 299F and 444LF is followed in a Spectramax Fluorimeter at 360 nm excitation and 440 nm emission. Hydrolysis of substrate 251 and S-2288 is followed in a Spectramax Spectrophotometer at 405 nm.

All assays are performed in a buffer consisting of 50 mM Hepes pH 7.4, 100 mM NaCl, 5 mM CaCl2, 0.01% Tween80. Inhibitors are used at 10, 20, 50, 100, 200 μM concentration. The FIXa assay is performed using 100 μM substrate, the tPA assay is performed using 10 μM substrate.

### (2)_{b} Amidolytic Assays for FXIa and FXIIa

The enzymes FXIa and FXIIa are from American Diagnostica; FXIa (cat no 4011a), FXIIa (cat no 412HA) and trypsin (cat no 20465) are from Life Technology. The chromogenic substrates (Chromogenix) in use are 2288 for FXIa and 2765 for FXIIa.

Hydrolysis of the chromogenic substrates is followed in a Spectramax Spectrophotometer at 405 nm for 10-20 min with intervals of 5-20 sec depending on the enzyme.

All assays are performed in a buffer consisting of 50 mM Hepes pH 7.4, 100 mM NaCl, 5 mM CaCl2, 0.01% BSA. With the exception that for the FIXa assays ethylene glycol is further added to a final concentration of 40%. In the assays substrate concentrations of 50, 100, 200, 500, 1000 μM are employed for each inhibitor concentration: 25, 50, 100, 500 μM.

### (2)c Data Analysis

For the assays run at a single substrate concentration, KI is determined using the formula VO/VI=1+I/KI (valid for S<<Km), by linear fitting of the values determined at several different inhibitor concentrations. VO is the rate of hydrolysis without inhibitors present, VI is the rate of hydrolysis at the in the presence of inhibitor and I is the inhibitor concentration.

From double reciprocal plots of 1/v versus 1/s for each inhibitor concentration the slope (Km(app)/V) is determined. This is followed by plots of Km(app)/V against the inhibitor concentration. Ki is determined as the intercept of the straight line at the i-axis.

### Stabilizing Effect of a Compound of Formula I on FVIIa

### Clot Assay/One-Stage Coagulation Assay:

The biological activity of a Factor VII polypeptide, such as FVIIa, may be measured using a one-stage coagulation assay. For this purpose, the sample to be tested is diluted in 50 mM PIPES-buffer (pH 7.5), 0.1% BSA, and 40 μl is incubated with 40 μl of Factor VII deficient plasma and 80 μl of human recombinant tissue factor containing 10 mM Ca²⁺ and synthetic phospholipids. Coagulation times are measured and compared to a standard curve using a reference standard in a parallel line assay.

In order to investigate the effect of a chosen compound of formula I on the stability of rhFVIIa, formulations such as the following may typically be prepared:

### Formulation 1:

1.0 mg/mL rhFVIIa (produced by Novo Nordisk A/S, Denmark)
10-20 mM histidine
10 mM glycylglycine
50 mM sodium chloride
10 mM calcium chloride
1 mM test compound (compound of formula I)
pH=6.5

### Formulation 2:

1.0 mg/mL rhFVIIa
10-20 mM histidine
10 mM glycylglycine
50 mM sodium chloride
10 mM calcium chloride
pH=6.5

The formulations are suitably prepared by mixing of a rhFVIIa bulk solution, a placebo solution of the excipients and a stock solution of the test compound to obtain the above-mentioned concentrations. The pH is suitably adjusted to a final pH of 6.5, e.g. using 1 M sodium hydroxide/hydrogen chloride.

The formulations are suitably stored at two temperatures, e.g. at 5° C. and at a temperature in the range of 25-30° C., respectively, and the analyses for clot activity are performed at appropriate time points, e.g. after ½ month, 1 month and thereafter at monthly intervals for both storage temperatures.

1. A compound of the formula I: 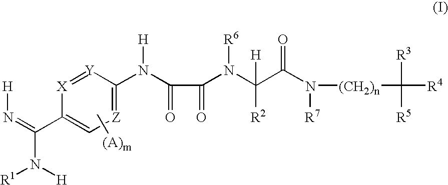
wherein
m is 0, 1 or 2;
n is 0 or 1;
A is halogen or hydroxy;
X, Y and Z independently are carbon or nitrogen;
or
X and Y taken together (i.e. the moiety X═Y) is a sulfur atom, and Z is carbon or nitrogen;
R¹ is chosen from hydrogen, hydroxy, (C₁-C₁₂)-alkoxycarbonyl-, (C₆-C₁₄)-aryl-(C₁-C₄)-alkoxycarbonyl-, and (C₆-C₁₄)-aryloxycarbonyl-, wherein each of the aryl groups is unsubstituted or substituted by at least one identical or different substituent chosen from (C₁-C₁₂)-alkyl, halogen, and (C₁-C₁₂)-alkoxy;
R² is chosen from hydrogen, (C₁-C₁₂)-alkyl, (C₆-C₁₄)-aryl, (C₆-C₁₄)-aryl-(C₁-C₄)-alkyl-,
R²⁰—(C₁-C₁₂)-alkyl-, R²⁰—(C₆-C₁₄)-aryl-, and R²⁰—(C₆-C₁₄)-aryl-(C₁-C₄)-alkyl-, wherein R²⁰ is chosen from hydroxycarbonyl-, aminocarbonyl-, (C₁-C₁₂)-alkoxycarbonyl-, and (C₆-C₁₄)-aryl-(C₁-C₄)-alkoxycarbonyl-;
R³ is chosen from hydrogen, cyano, hydroxy, and (C₁-C₁₂)-alkyl;
R⁴ is chosen from (C₁-C₁₂)-alkyl, (C₆-C₁₄)-aryl, (C₆-C₁₄)-aryl-(C₁-C₄)-alkyl-, Het, and Het-(C₁-C₄)-alkyl-, wherein the alkyl, aryl and Het groups are unsubstituted or substituted by at least one identical or different substituent R¹⁰;
R⁵ is chosen from hydrogen, (C₁-C₁₂)-alkyl, (C₆-C₁₄)-aryl, (C₆-C₁₄)-aryl-(C₁-C₄)-alkyl-, Het, Het-(C₁-C₄)-alkyl-, (C₆-C₁₄)-aryl-(C₁-C₄)-alkyl-aminocarbonyl-, and Het-(C₁-C₄)-alkyl-aminocarbonyl-, wherein the alkyl, aryl and Het groups are unsubstituted or substituted by at least one identical or different substituent R¹⁰;
R⁶ and R⁷ independently are chosen from hydrogen and (C₁-C₈)-alkyl;
R¹⁰ is chosen from (C₁-C₁₂)-alkyl, (C₆-C₁₄)-aryl-(C₁-C₄)-alkyl-, (C₁-C₈)-alkoxy, (C₁-C₄)-alkoxy-(C₂-C₄)-alkoxy-, (C₆-C₁₄)-aryl-(C₁-C₄)-alkoxy-, (C₆-C₁₄)-aryloxy-, Het- oxy-, Het-(C₁-C₄)-alkoxy-, (C₆-C₁₄)-aryl, Het, Het-(C₁-C₄)-alkyl-, trifluoromethoxy, trifluoromethyl, halogen, oxo, hydroxy, amino, (C₁-C₁₂)-alkylcarbonylamino-, aminocarbonylamino-, (C₆-C₁₄)-arylcarbonylamino-, Het-carbonylamino-, (C₆-C₁₄)-aryl(C₁-C₄)-alkylcarbonylamino-, Het-(C₁-C₄)-alkylcarbonylamino-, (C₁-C₈)-alkylcarbonyl-, (C₆-C₁₄)-arylcarbonyl-, (C₁-C₈)-alkylaminocarbonyl-, (C₆-C₁₄)-arylaminocarbonyl-, (C₆-C₁₄)-aryl-(C₁-C₄)-alkylaminocarbonyl-, Het-aminocarbonyl-, Het-(C₁-C₄)-alkylaminocarbonyl-, aminocarbonyl-, (C₁-C₈)-alkoxycarbonyl-, hydroxycarbonyl-, cyano, nitro, amidino, acetimino, tri-((C₁-C₄)-alkyl)ammonio-, (C₁-C₈)-alkylamino-, di-((C₁-C₈)-alkyl)amino-, hydroxycarbonylmethoxy-, (C₁-C₈)-alkylsulfonyl-, (C₆-C₁₄)-arylsulfonyl-, (C₁-C₈)-alkylaminosulfonyl-, (C₆-C₁₄)-arylaminosulfonyl-, (C₆-C₁₄)-aryl(C₁-C₄)-alkylaminosulfonyl-, Het-aminosulfonyl-, Het-(C₁-C₄)-alkylaminosulfonyl-, (C₁-C₈)-alkylsulfonylamino-, (C₆-C₁₄)-arylsulfonylamino-, (C₆-C₁₄)-aryl-(C₁-C₄)-alkylsulfonylamino-, Het-sulfonylamino-, and Het-(C₁-C₄)-alkylsulfonylamino-, wherein (C₁-C₁₂)-alkylcarbonylamino-representing R¹⁰ is unsubstituted or substituted in the alkyl group by a substituent chosen from amino, hydroxy and (C₁-C₄)-alkoxy, and wherein (C₁-C₁₂)-alkyl and (C₁-C₈)-alkoxy representing R¹⁰ are unsubstituted or substituted by at least one identical or different substituent chosen from (C₁-C₈)-alkoxycarbonyl-, hydroxycarbonyl-, and aminocarbonyl-,
wherein each of the aryl groups and Het group in a group R¹⁰ is unsubstituted or substituted by at least one identical or different substituent chosen from halogen, nitro, oxo, hydroxy, (C₁-C₈)-alkyl, (C₁-C₈)-alkoxy, (C₁-C₄)-alkoxy-(C₂-C₄)-alkoxy-, (C₆-C₁₄)-aryloxy-, (C₆-C₁₄)-aryl-(C₁-C₄)-alkoxy-, Het-oxy-, Het-(C₁-C₄)-alkoxy-, (C₆-C₁₄)-aryl, (C₆-C₁₄)-aryl-(C₁-C₄)-alkyl-, Het, Het-(C₁-C₄)-alkyl-, trifluoromethyl, cyano, trifluoromethoxy, (C₁-C₈)-alkylsulfonyl-, (C₁-C₈)-alkoxycarbonyl-, hydroxycarbonyl-, aminocarbonyl-, amino, (C₁-C₈)-alkylamino-, di-((C₁-C₈)-alkyl)amino-, (C₁-C₈)-alkylcarbonylamino-, (C₆-C₁₄)-aryl-(C₁-C₄)-alkylcarbonylamino-, (C₆-C₁₄)-arylcarbonylamino-, Het-carbonylamino-, Het-(C₁-C₄)-alkylcarbonylamino-, and (C₁-C₈)-alkylcarbonyl-, wherein (C₁-C₈)-alkyl and (C₁-C₈)-alkoxy representing a substituent on an aryl group or Het group in a group R¹⁰ are unsubstituted or substituted by at least one identical or different substituent chosen from (C₁-C₈)-alkoxycarbonyl-, hydroxycarbonyl-, and aminocarbonyl-;
Het is a residue of a saturated or unsaturated monocyclic or bicyclic, 3-membered to 10-membered heterocyclic ring system containing 1, 2 or 3 identical or different ring heteroatoms chosen from nitrogen, oxygen and sulfur;
including any and all stereoisomeric form or forms thereof;
and any mixture of two or more such compounds of formula I in any ratio;
and physiologically tolerable salts thereof.

2. A compound according to claim 1 having the formula Ia: 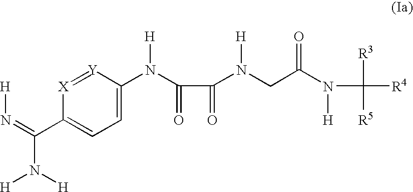
wherein X, Y, R³, R⁴ and R⁵ are as defined in claim 1,
including any and all stereoisomeric form or forms thereof;
and any mixture of two or more such compounds of formula Ia in any ratio;
and physiologically tolerable salts thereof.

3. A pharmaceutical composition comprising: one or more compounds of the formula I: 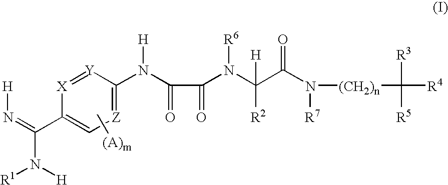
wherein
m is 0, 1 or 2;
n is 0 or 1;
A is halogen or hydroxy;
X, Y and Z independently are carbon or nitrogen;
or
X and Y taken together (i.e. the moiety X═Y) is a sulfur atom, and Z is carbon or nitrogen;
R¹ is chosen from hydrogen hydroxy, (C₁-C₁₂)-alkoxycarbonyl-, (C₆-C₁₄)-aryl-(C₁-C₄)-alkoxycarbonyl-, and (C₆-C₁₄)-aryloxycarbonyl-, wherein each of the aryl groups is unsubstituted or substituted by at least one identical or different substituent chosen from (C₁-C₁₂)-alkyl, halogen and (C₁-C₁₂)-alkoxy;
R² is chosen from hydrogen, (C₁-C₁₂)-alkyl, (C₆-C₁₄)-aryl (C₆-C₁₄)-aryl-(C₁-C₄)-alkyl-,
R²⁰—(C₁-C₁₂)-alkyl-, R²⁰—(C₁-C₁₄)-aryl-, and R²⁰—(C₆-C₁₄)-aryl-(C₁-C₄)-alkyl-, wherein R²⁰ is chosen from hydroxycarbonyl-, aminocarbonyl-, (C₁-C₁₂)-alkoxycarbonyl-, and (C₆-C₁₄)-aryl-(C₁-C₄)-alkoxycarbonyl-;
R³ is chosen from hydrogen, cyano, hydroxy, and (C₁-C₁₂)-alkyl;
R⁴ is chosen from (C₁-C₁₂)-alkyl, (C₆-C₁₄)-aryl, (C₆-C₁₄)-aryl-(C₁-C₄)-alkyl-, Het, and Het-(C₁-C₄)-alkyl-, wherein the alkyl, aryl and Het groups are unsubstituted or substituted by at least one identical or different substituent R¹⁰;
R⁵ is chosen from hydrogen, (C₁-C₁₂)-alkyl,(C₆-C₁₄)-aryl, (C₁-C₁₄)-aryl-(C₁-C₄)-alkyl-, Het, Het-(C₁-C₄)-alkyl-, (C₆-C₁₄)-aryl-(C₁-C₄)-alkyl-aminocarbonyl-, and Het-(C₁-C₄)-alkyl-aminocarbonyl-, wherein the alkyl, aryl and Het groups are unsubstituted or substituted by at least one identical or different substituent R¹⁰;
R⁶ and R⁷ independently are chosen from hydrogen and (C₁-C₈)-alkyl;
R¹⁰ is chosen from (C₁-C₁₂)-alkyl, (C₆-C₁₄)-aryl-(C₁-C₄)-alkyl-, (C₁-C₈)-alkoxy, (C₁-C₄)-alkoxy-(C₂-C₄)-alkoxy-, (C₆-C₁₄)-aryl-(C₁-C₄)-alkoxy-, (C₆-C₁₄)-aryloxy-, Het-oxy-, Het-(C₁-C₄)-alkoxy-, (C₆-C₁₄)-aryl, Het, Het-(C₁-C₄)-alkyl-, trifluoromethoxy, trifluoromethyl, halogen, oxo, hydroxy, amino, (C₁-C₁₂)-alkylcarbonylamino-, aminocarbonylamino-, (C₆-C₁₄)-arylcarbonylamino-, Het-carbonylamino-, (C₆-C₁₄)-aryl-(C₁-C₄)-alkylcarbonylamino-, Het-(C₁-C₄)-alkylcarbonylamino-, (C₁-C₈)-alkylcarbonyl-, (C₆-C₁₄)-arylcarbonyl-, (C₁-C₈)-alkylaminocarbonyl-, (C₆-C₁₄)-arylaminocarbonyl-, (C₆-C₁₄)-aryl-(C₁-C₄)-alkylaminocarbonyl-, Het-aminocarbonyl-, Het-(C₁-C₄)-alkylaminocarbonyl-, aminocarbonyl-, (C₁-C₈)-alkoxycarbonyl-, hydroxycarbonyl-, cyano, nitro, amidino, acetimino, tri-((C₁-C₄)-alkyl)ammonio-, (C₁-C₈)-alkylamino-, di-((C₁-C₈)-alkyl)amino-, hydroxycarbonylmethoxy-(C₁-C₈)-alkylsulfonyl-, (C₆-C₁₄)-arylsulfonyl-, (C₁-C₈)-alkylaminosulfonyl-L (C₆-C₁₄)-arylaminosulfonyl-, (C₆-C₁₄)-aryl(C₁-C₄)-alkylaminosulfonyl-, Het-aminosulfonyl-, Het-(C₁-C₄)-alkylaminosulfonyl-, (C₁-C₈)-alkylsulfonylamino-, (C₆-C₄)-arylsulfonylamino-, (C₆-C₁₄)-aryl-(C₁-C₄)-alkylsulfonylamino-, Het-sulfonylamino-, and Het-(C₁-C₄)-alkylsulfonylamino-, wherein (C₁-C₁₂)-alkylcarbonylamino- representing R¹⁰ is unsubstituted or substituted in the alkyl group by a substituent chosen from amino, hydroxy and (C₁-C₄)-alkoxy, and wherein (C₁-C₁₂)-alkyl and (C₁-C₈)-alkoxy representing R¹⁰ are unsubstituted or substituted by at least one identical or different substituent chosen from (C₁-C₈)-alkoxycarbonyl-, hydroxycarbonyl-, and aminocarbonyl-,
wherein each of the aryl groups and Het group in a group R¹⁰ is unsubstituted or substituted by at least one identical or different substituent chosen from halogen, nitro oxo, hydroxy, (C₁-C₈)-alkyl, (C₁-C₈)-alkoxy, (C₁-C₄)-alkoxy-(C₁-C₄)-alkoxy-, (C₆-C₁₄)-aryloxy-, (C₆-C₁₄)-aryl-(C₁-C₄)-alkoxy-, Het-oxy-, Het-(C₁-C₄)-alkoxy-, (C₆-C₁₄)-aryl, (C₆-C₁₄)-aryl-(C₁-C₄)-alkyl-, Het, Het-(C₁-C₄)-alkyl-, trifluoromethyl, cyano, trifluoromethoxy, (C₁-C₈)-alkylsulfonyl-, (C₁-C₈)-alkoxycarbonyl-, hydroxycarbonyl-, aminocarbonyl-, amino, (C₁-C₈)-alkylamino-, di-((C₁-C₈)-alkyl)amino-, (C₁-C₈)-alkylcarbonylamino-, (C₆-C₁₄)-aryl-(C₁-C₄)-alkylcarbonylamino-, (C₁-C₁₄)-arylcarbonylamino-, Het-carbonylamino-, Het-(C₁-C₄)-alkylcarbonylamino-, and (C₁-C₈)-alkylcarbonyl-, wherein (C₁-C₈)-alkyl and (C₁-C₈)-alkoxy representing a substituent on an aryl group or Het group in a group R¹⁰ are unsubstituted or substituted by at least one identical or different substituent chosen from (C₁-C₈)-alkoxycarbonyl-, hydroxycarbonyl-, and aminocarbonyl-;
Het is a residue of a saturated or unsaturated monocyclic or bicyclic, 3-membered to 10-membered heterocyclic ring system containing 1, 2 or 3 identical or different ring heteroatoms chosen from nitrogen, oxygen and sulfur;
including any and all stereoisomeric form or forms thereof;
and any mixture of two or more such compounds of formula I in any ratio, or physiologically tolerable salts thereof; and a Factor VII polypeptide.

4. A pharmaceutical composition according to claim 3, wherein said Factor VII polypeptide is selected from: wild-type human Factor VIIa; Factor VII variants; and Factor VII derivatives.

5. A pharmaceutical composition according to claim 3, further comprising a pharmaceutically acceptable carrier or diluent.

6. A pharmaceutical composition according to claim 3 which is a liquid, aqueous composition.

7. A method of preparing a composition comprising a Factor VII polypeptide, comprising: adding a compound of the formula I: 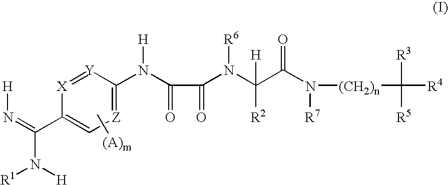
wherein
m is 0, 1 or 2;
n is 0 or 1;
A is halogen or hydroxy;
X, Y and Z independently are carbon or nitrogen;
or
X and Y taken together (i.e. the moiety X═Y) is a sulfur atom and Z is carbon or nitrogen;
R¹ is chosen from hydrogen, hydroxy, (C₁-C₁₂)-alkoxycarbonyl-, (C₆-C₁₄)-aryl-(C₁-C₄)-alkoxycarbonyl-, and (C₆-C₁₄)-aryloxycarbonyl-, wherein each of the aryl groups is unsubstituted or substituted by at least one identical or different substituent chosen from (C₁-C₁₂)-alkyl, halogen, and (C₁-C₁₂)-alkoxy;
R² is chosen from hydrogen, (C₁-C₁₂)-alkyl, (C₆-C₁₄)-aryl, (C₆-C₁₄)-aryl-(C₁-C₄)-alkyl-,
R²⁰—(C₁-C₁₂)-alkyl-, R²⁰—(C₆-C₁₄)-aryl-, and R²⁰—(C₆-C₁₄)-aryl-(C₁-C₄)-alkyl-, wherein R²⁰ is chosen from hydroxycarbonyl-, aminocarbonyl-, (C₁-C₁₂)-alkoxycarbonyl-, and (C₆-C₁₄)-aryl-(C₁-C₄)-alkoxycarbonyl-;
R³ is chosen from hydrogen cyano hydroxy and (C₁-C₁₂)-alkyl;
R⁴ is chosen from (C₁-C₁₂)-alkyl, (C₆-C₁₄)-aryl-(C₆-C₁₄-aryl-(C₁-C₄)-alkyl-, Het, and Het-(C₁-C₄)-alkyl-, wherein the alkyl, aryl and Het groups are unsubstituted or substituted by at least one identical or different substituent R¹⁰;
R⁵ is chosen from hydrogen (C₁-C₁₂)-alkyl, (C₆-C₁₄)-aryl, (C₆-C₁₄)-aryl-(C₁-C₄)-alkyl-, Het, Het-(C₁-C₄)-alkyl-, (C₆-C₁₄)-aryl-(C₁-C₄)-alkyl-aminocarbonyl-, and Het-(C₁-C₄)-alkyl-aminocarbonyl-, wherein the alkyl, aryl and Het groups are unsubstituted or substituted by at least one identical or different substituent R¹⁰;
R⁶ and R⁷ independently are chosen from hydrogen and (C₁-C₈)-alkyl;
R¹⁰ is chosen from (C₁-C₁₂)-alkyl, (C₆-C₁₄)-aryl-(C₁-C₄)-alkyl-, (C₁-C₈)-alkoxy, (C₁-C₄)-alkoxy-(C₁-C₄)-alkoxy-, (C₆-C₁₄)-aryl-(C₁-C₄)-alkoxy-, (C₆-C₁₄)-aryloxy-, Het-oxy-, Het-(C₁-C₄)-alkoxy-, (C₆-C₁₄)-aryl, Het, Het-(C₁-C₄)-alkyl-, trifluoromethoxy, trifluoromethyl, halogen, oxo, hydroxy amino, (C₁-C₁₂)-alkylcarbonylamino-, aminocarbonylamino-, (C₆-C₁₄)-arylcarbonylamino-, Het-carbonylamino-, (C₆-C₁₄)-aryl(C₁-C₄)-alkylcarbonylamino-, Het-(C₁-C₄)-alkylcarbonylamino-, (C₁-C₈)-alkylcarbonyl-, (C₆-C₁₄)-arylcarbonyl-, (C₁-C₈)-alkylaminocarbonyl-, (C₆-C₁₄)-arylaminocarbonyl-, (C₆-C₁₄)-aryl-(C₁-C₄)-alkylaminocarbonyl-, Het-aminocarbonyl-, Het-(C₁-C₄)-alkylaminocarbonyl-, aminocarbonyl-, (C₁-C₈)-alkoxycarbonyl-, hydroxycarbonyl-, cyano, nitro, amidino, acetimino, tri-((C₁-C₄)-alkyl)ammonio-, (C₁-C₈)-alkylamino-, di-((C₁-C₈)-alkyl)amino-, hydroxycarbonylmethoxy-, (C₁-C₈)-alkylsulfonyl-, (C₆-C₁₄)-arylsulfonyl-, (C₁-C₈)-alkylaminosulfonyl-, (C₆-C₁₄)-arylaminosulfonyl-, (C₆-C₁₄)-aryl(C₁-C₄)-alkylaminosulfonyl-, Het-aminosulfonyl-, Het-(C₁-C₄)-alkylaminosulfonyl-, (C₁-C₈)-alkylsulfonylamino-, (C₆-C₁₄)-arylsulfonylamino-, (C₆-C₁₄)-aryl-(C₁-C₄)-alkylsulfonylamino-, Het-sulfonylamino-, and Het-(C₁-C₄)-alkylsulfonylamino-, wherein (C₁-C₁₂)-alkylcarbonylamino- representing R¹⁰ is unsubstituted or substituted in the alkyl group by a substituent chosen from amino, hydroxy and (C₁-C₄)-alkoxy, and wherein (C₁-C₁₂)-alkyl and (C₁-C₈)-alkoxy representing R¹⁰ are unsubstituted or substituted by at least one identical or different substituent chosen from (C₁-C₈)-alkoxycarbonyl-, hydroxycarbonyl-, and aminocarbonyl-,
wherein each of the aryl groups and Het group in a group R¹⁰ is unsubstituted or substituted by at least one identical or different substituent chosen from halogen nitro oxo, hydroxy, (C₁-C₈)-alkyl, (C₁-C₈)-alkoxy, (C₁-C₄)-alkoxy-(C₂-C₄)-alkoxy-,(C₆-C₁₄)-aryloxy-, (C₆-C₁₄)-aryl-(C₁-C₄)-alkoxy-, Het-oxy-, Het-(C₁-C₄)-alkoxy-, (C₆-C₁₄)-aryl, (C₆-C₁₄)-aryl-(C₁-C₄)-alkyl-, Het, Het-(C₁-C₄)-alkyl-, trifluoromethyl, cyano, trifluoromethoxy, (C₁-C₈)-alkylsulfonyl-, (C₁-C₈)-alkoxycarbonyl-, hydroxycarbonyl-, aminocarbonyl-, amino, (C₁-C₈)-alkylamino-, di-((C₁-C₈)-alkyl)amino-, (C₁-C₈)-alkylcarbonylamino-, (C₆-C₁₄)-aryl-(C₁-C₄)-alkylcarbonylamino-, (C₆-C₁₄)-arylcarbonylamino-, Het-carbonylamino-, Het-(C₁-C₄)-alkylcarbonylamino-, and (C₁-C₈)-alkylcarbonyl-, wherein (C₁-C₈)-alkyl and (C₁-C₈)-alkoxy representing a substituent on an aryl group or Het group in a group R¹⁰ are unsubstituted or substituted by at least one identical or different substituent chosen from (C₁-C₈)-alkoxycarbonyl-, hydroxycarbonyl-, and aminocarbonyl-;
Het is a residue of a saturated or unsaturated monocyclic or bicyclic, 3-membered to 10-membered heterocyclic ring system containing 1, 2 or 3 identical or different ring heteroatoms chosen from nitrogen, oxygen and sulfur;
including any and all stereoisomeric form or forms thereof;
and any mixture of two or more such compounds of formula I in any ratio, or, or a physiologically tolerable salt thereof, to a sample containing said Factor VII polypeptide; or adding said Factor VII polypeptide to a sample containing said compound, or a physiologically tolerable salt thereof.

8. A method according to claim 7, wherein said Factor VII polypeptide is selected from: wild-type human Factor VIIa; Factor VII variants; and Factor VII derivatives.

9. A method according to claim 7, wherein said compound or salt thereof and/or said Factor VII polypeptide is present in a liquid, aqueous medium.

10. A pharmaceutical composition prepared by a method according to claim 7.

11. A method of inhibiting a Factor VII polypeptide, comprising: adding a compound of the formula I: 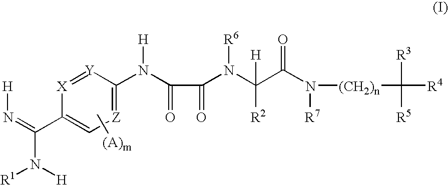
wherein
m is 0, 1 or 2;
n is 0 or 1;
A is halogen or hydroxy;
X, Y and Z independently are carbon or nitrogen;
or
X and Y taken together (i.e. the moiety X═Y) is a sulfur atom and Z is carbon or nitrogen;
R¹ is chosen from hydrogen hydroxy, (C₁-C₁₂)-alkoxycarbonyl-, (C₆-C₁₄)-aryl-(C₁-C₄)-alkoxycarbonyl-, and (C₆-C₁₄)-aryloxycarbonyl-, wherein each of the aryl groups is unsubstituted or substituted by at least one identical or different substituent chosen from (C₁-C₁₂)-alkyl, halogen, and (C₁-C₁₂)-alkoxy;
R² is chosen from hydrogen (C₁-C₁₂)-alkyl, (C₆-C₁₄)-aryl, (C₆-C₁₄)-aryl-(C₁-C₄)-alkyl-,
R²⁰—(C₁-C₁₂)-alkyl-, R²⁰—(C₆-C₁₄)-aryl-, and R²⁰—(C₆-C₁₄)-aryl-(C₁-C₄)-alkyl-, wherein R²⁰ is chosen from hydroxycarbonyl-, aminocarbonyl-, (C₁-C₁₂)-alkoxycarbonyl-, and (C₆-C₁₄)-aryl-(C₁-C₄)-alkoxycarbonyl-;
R³ is chosen from hydrogen, cyano, hydroxy, and (C₁-C₁₂)-alkyl;
R⁴ is chosen from (C₁-C₁₂)-alkyl, (C₆-C₁₄)-aryl, (C₆-C₁₄)-aryl-(C₁-C₄)-alkyl-, Het, and Het-(C₁-C₄)-alkyl-, wherein the alkyl, aryl and Het groups are unsubstituted or substituted by at least one identical or different substituent R¹⁰;
R⁵ is chosen from hydrogen, (C₁-C₁₂)-alkyl, (C₆-C₁₄)-aryl, (C₆-C₁₄)-aryl-(C₁-C₄) -alkyl-, Het, Het-(C₁-C₄)-alkyl-, (C₆-C₁₄)-aryl-(C₁-C₄)-alkyl-aminocarbonyl-, and Het-(C₁-C₄)-alkyl-aminocarbonyl-, wherein the alkyl aryl and Het groups are unsubstituted or substituted by at least one identical or different substituent R¹⁰;
R⁶ and R⁷ independently are chosen from hydrogen and (C₁-C₈)-alkyl;
R¹⁰ is chosen from (C₁-C₁₂)-alkyl, (C₆-C₁₄)-alkyl-(C₁-C₄)-alkyl-, (C₁-C₈)-alkoxy, (C₁-C₄)-alkoxy-(C₂-C₄)-alkoxy-, (C₆-C₁₄)-aryl-(C₁-C₄)-alkoxy-, (C₆-C₁₄)-aryloxy-, Het-oxy-, Het-(C₁-C₄)-alkoxy-, (C₆-C₁₄)-aryl, Het, Het-(C₁-C₄)-alkyl-, trifluoromethoxy trifluoromethyl, halogen, oxo, hydroxy amino. (C₁-C₁₂)-alkylcarbonylamino-, aminocarbonylamino-, (C₆-C₁₄)-arylcarbonylamino-, Het-carbonylamino-, (C₆-C₁₄)-aryl(C₁-C₄)-alkylcarbonylamino-, Het-(C₁-C₄)-alkylcarbonylamino-, (C₁-C₈)-alkylcarbonyl-, (C₆-C₁₄)-arylcarbonyl-, (C₁-C₈)-alkylaminocarbonyl-, (C₆-C₁₄)-arylaminocarbonyl-, (C₆-C₁₄)-aryl-(C₁-C₄)-alkylaminocarbonyl-, Het-aminocarbonyl-, Het-(C₁-C₄)-alkylaminocarbonyl-, aminocarbonyl-, (C₁-C₈)-alkoxycarbonyl-, hydroxycarbonyl-, cyano, nitro, amidino, acetimino, tri-((C₁-C₄)-alkyl)ammonio-, (C₁-C₈)-alkylamino-, di-((C₁-C₈)-alkyl)amino-, hydroxycarbonylmethoxy-, (C₁-C₈)-alkylsulfonyl-, (C₆-C₁₄)-arylsulfonyl-, (C₁-C₈)-alkylaminosulfonyl-, (C₆-C₁₄)-arylaminosulfonyl-, (C₆-C₁₄)-aryl(C₁-C₄)-alkylaminosulfonyl-, Het-aminosulfonyl-, Het-(C₁-C₄)-alkylaminosulfonyl-, (C₁-C₈)-alkylsulfonylamino-, (C₆-C₁₄)-arylsulfonylamino-, (C₆-C₁₄)-aryl-(C₁-C₄)-alkylsulfonylamino-, Het-sulfonylamino-, and Het-(C₁-C₄)-alkylsulfonylamino-, wherein (C₁-C₁₂)-alkylcarbonylamino- representing R¹⁰ is unsubstituted or substituted in the alkyl group by a substituent chosen from amino, hydroxy and (C₁-C₄)-alkoxy and wherein (C₁-C₁₂)-alkyl and (C₁-C₈)-alkoxy representing R¹⁰ are unsubstituted or substituted by at least one identical or different substituent chosen from (C₁-C₈)-alkoxycarbonyl-, hydroxycarbonyl-, and aminocarbonyl-,
wherein each of the aryl groups and Het group in a group R¹⁰ is unsubstituted or substituted by at least one identical or different substituent chosen from halogen, nitro oxo, hydroxy, (C₁-C₈)-alkyl, (C₁-C₈)-alkoxy, (C₁-C₄)-alkoxy-(C₂-C₄)-alkoxy-, (C₆-C₁₄)-aryloxy-, (C₆-C₁₄)-aryl-(C₁-C₄)-alkoxy-, Het-oxy-, Het-(C₁-C₄)-alkoxy-, (C₆-C₁₄)-aryl, (C₆-C₁₄)-arly-(C₁-C₄)-alkyl-, Het, Het-(C₁-C₄)-alkyl-, trifluoromethyl, cyano, trifluoromethoxy, (C₁-C₈)-alkylsulfonyl-, (C₁-C₈)-alkoxycarbonyl-, hydroxycarbonyl-, aminocarbonyl-, amino, (C₁-C₈)-alkylamino-, di-((C₁-C₈)-alkyl)amino-, (C₁-C₈)-alkylcarbonylamino-, (C₆-C₁₄)-aryl-(C₁-C₄)-alkylcarbonylamino-, (C₆-C₁₄)-arylcarbonylamino-, Het-carbonylamino-, Het-(C₁-C₄)-alkylcarbonylamino-, and (C₁-C₈)-alkylcarbonyl-, wherein (C₁-C₈)-alkyl and (C₁-C₈)-alkoxy representing a substituent on an aryl group or Het group in a group R¹⁰ are unsubstituted or substituted by at least one identical or different substituent chosen from (C₁-C₈)-alkoxycarbonyl-, hydroxycarbonyl-, and aminocarbonyl-;
Het is a residue of a saturated or unsaturated monocyclic or bicyclic, 3-membered to 10-membered heterocyclic ring system containing 1, 2 or 3 identical or different ring heteroatoms chosen from nitrogen oxygen and sulfur;
including any and all stereoisomeric form or forms thereof;
and any mixture of two or more such compounds of formula I in any ratio, or physiologically tolerable salts thereof, to a sample containing said Factor VII polypeptide; or adding said Factor VII polypeptide to a sample containing said compound, or a physiologically tolerable salt thereof.

12. A method according to claim 11, wherein said Factor VII polypeptide is selected from: wild-type human Factor VIIa; Factor VII variants; and Factor VII derivatives.

13. A method according to claim 11, wherein said compound or salt thereof and/or said Factor VII polypeptide is present in a liquid, aqueous medium.

14. (canceled)
